(19) United States
(12) Patent Application Publication
(10) Pub. No.: US 2015/0230751 A1
(43) Pub. Date: Aug. 20, 2015

(54) INFORMATION MANAGEMENT APPARATUS, INFORMATION MANAGEMENT METHOD, INFORMATION MANAGEMENT SYSTEM, STETHOSCOPE, INFORMATION MANAGEMENT PROGRAM, MEASUREMENT SYSTEM, CONTROL PROGRAM, AND RECORDING MEDIUM

(71) Applicant: Sharp Kabushiki Kaisha, Osaka-shi, Osaka (JP)

(72) Inventors: Mikihiro Yamanaka, Osaka-shi (JP); Tomohisa Kawata, Osaka-shi (JP); Yutaka Ikeda, Osaka-shi (JP)

(21) Appl. No.: 14/363,482

(22) PCT Filed: Dec. 10, 2012
(86) PCT No.: PCT/JP2012/081959

§ 371 Date: Jun. 6, 2014

(30) Foreign Application Priority Data
Dec. 13, 2011 (JP) 2011-272782
Dec. 13, 2011 (JP) 2011-272783

(51) Int. Cl. A61B 5/00; A61B 7/04

(52) U.S. Cl. CPC A61B 5/684; A61B 7/04; A61B 5/749; A61B 5/7246; A61B 5/7278; A61B 5/742

(57) ABSTRACT

In order to easily associate body sound information with measurement position information, a terminal device (30) includes an information manager (35) that associates body sound information obtained by a stethoscope (1) with position information indicating a position at which the body sound information has been obtained. The position information is information input into a sound collector and is input into the terminal device (30) as voice.

### TECHNICAL FIELD

The present invention relates to a stethoscope for obtaining body sounds and to an information management apparatus, an information management system, and an information management method for managing measurement information concerning body sounds (body sound information) and so on.

### BACKGROUND ART

When obtaining body sounds (such as breath sounds or heartbeats) of a patient by using a stethoscope, position information indicating a portion of a body surface of a patient from which body sounds have been obtained by applying the stethoscope to this portion is important. Body sounds are location-dependent, and obtained body sounds are different depending on where a stethoscope is applied.

PTL 1 discloses that an auscultation sound signal indicating obtained auscultation sounds is recorded as one file and appropriate-point identification data indicating a preset appropriate point on a body surface of a patient is added to this file.

PTL 2 discloses that, while observing captured images of a patient, a physician being in a remote site specifies a position at which an auscultation microphone will be applied.

PTL 3 discloses that, while the operating state for using a medical device by a user is being monitored by a camera and the behavior of the user is being expressed on a graphic in real time, corrections for errors are requested in a remote site.

### CITATION LIST

### Patent Literature

PTL 1: Japanese Unexamined Patent Application Publication No. 2001-327488 (publication date: Nov. 27, 2001)

PTL 2: Japanese Unexamined Patent Application Publication No. 2008-113936 (publication date: May 22, 2008)

PTL 3: Japanese Unexamined Patent Application Publication (Translation of PCT Application) No. 2010-525363 (publication date: Jul. 22, 2010)

PTL 4: Japanese Unexamined Patent Application Publication No. 2005-111260 (publication date: Apr. 28, 2005)

PTL 5: Japanese Unexamined Patent Application Publication No. 2005-40178 (publication date: Feb. 17, 2005)

### SUMMARY OF INVENTION

### Technical Problem

In the invention disclosed in PTL 1, an auscultation sound signal indicating auscultation sounds which are continuously collected from when the recording of such auscultation sounds is started until it is finished by using an ON/OFF switch which controls the start and the end of recording of auscultation sounds is recorded as one file. Then, appropriate-point identification data is added to the file obtained in this manner in accordance with a switching operation of the ON/OFF switch. This increases the complexity of a recording operation for auscultation sounds.

Neither of PTL 2 nor PTL 3 discloses a configuration in which body sound information and measurement position information are simply associated with each other.

The present invention has been made in order to solve the above-described problems. It is an object of the present invention to provide an information management apparatus that is capable of simply associating body sound information with measurement position information, and also to provide an information management method, an information management system, and a stethoscope.

### Solution to Problem

In order to solve the above-described problems, an embodiment of the present invention provides an information management apparatus including: obtaining means for obtaining body sound information obtained by a sound collector and position information indicating a position at which the body sound information has been obtained; and associating means for associating the body sound information and the position information obtained by the obtaining means with each other. The obtaining means obtains the position information as voice input into the sound collector.

An information management method according to an embodiment of the present invention is an information management method for an information management apparatus. The information management method includes: a first obtaining step of obtaining body sound information obtained by a sound collector; a second obtaining step of obtaining position information indicating a position at which the body sound information has been obtained, as voice input into the sound collector; and an associating step of associating the body sound information obtained in the first obtaining step with the position information obtained in the second obtaining step.

A stethoscope according to an embodiment of the present invention includes: a first sound collecting unit that obtains body sounds; a second sound collecting unit that obtains, as voice information, position information indicating a position at which the body sounds have been obtained; and a transmitter that transmits body sound information indicating the body sounds and the position information to associating means for associating body sounds obtained by the first sound collecting unit with position information obtained by the second sound collecting unit.

In order to solve the above-described problems, the present invention provides an information management apparatus for managing body sound information collected by a stethoscope. The information management apparatus includes: subject information obtaining means for obtaining subject information concerning a subject from which body sound information is collected; an auscultation-assisting-information storage section that stores, according to the subject information, a plurality of patterns of auscultation assisting information each including at least one item of measurement position information indicating a measurement position to which a stethoscope will be applied; auscultation-assisting-information selecting means for selecting, from the plurality of patterns of auscultation assisting information stored in the auscultation-assisting-information storage section, one pattern of the auscultation assisting information corresponding to subject information obtained by the subject information obtaining means; output control means for generating an image signal from measurement position information included in the pattern of auscultation assisting information selected by the auscultation-assisting-information selecting means and for outputting the generated image signal; and information managing means for linking one item of the measurement position information output from the output control means to body sound information collected by the stethoscope. The information managing means specifies one item of the measurement position information on the basis of auxiliary information which has been obtained, as a trigger, upon the occurrence of a specific event while the image signal is being output.

In order to solve the above-described problems, the present invention provides an information management method for managing body sound information collected by a stethoscope. The information management method includes: a subject information obtaining step of obtaining subject information concerning a subject from which body sound information is collected; an auscultation-assisting-information selecting step of selecting one pattern of auscultation assisting information corresponding to subject information obtained in the subject information obtaining step, from a plurality of patterns of auscultation assisting information stored in an auscultation-assisting-information storage section according to the subject information, each of the plurality of patterns of auscultation assisting information including at least one item of measurement position information indicating a measurement position to which a stethoscope will be applied; an output control step of generating an image signal from measurement position information included in the pattern of auscultation assisting information selected in the auscultation-assisting-information selecting step and outputting the generated image signal; and an information managing step of linking one item of the measurement position information output in the output control step to body sound information collected by the stethoscope. The information managing step specifies one item of the measurement position information on the basis of auxiliary information which has been obtained, as a trigger, upon the occurrence of a specific event while the image signal is being output.

### Advantageous Effects of Invention

With the above-described configuration, the present invention achieves the advantages that the burden imposed on a user caused by inputting position information can be reduced and that the need to separately provide an input device for inputting position information can be eliminated.

With the above-described configuration, the present invention also achieves the advantages that it is possible to allow an operator to perform measurement by using a digital stethoscope without using a special device involving a complicated operation and that it is possible to easily link collected body sound information to measurement position information at which the body sound information has been obtained.

### BRIEF DESCRIPTION OF DRAWINGS

FIG. 1 illustrates the configuration of a body sound measurement system according to an embodiment of the present invention.

FIG. 2 illustrates an overview of a body sound measurement system according to an embodiment of the present invention.

FIG. 3 is a sectional view illustrating an example of the configuration of a chestpiece of a stethoscope included in the body sound measurement system.

FIG. 4 illustrates an example of an association table for associating body sound information with position information.

FIG. 5 is a flowchart illustrating an example of a flow of processing performed by the body sound measurement system.

FIG. 6 Parts (a) through (c) of FIG. 6 illustrate examples of screens displayed in a display unit of a terminal device included in the body sound measurement system.

FIG. 7 is a functional block diagram illustrating the configuration of the major parts of an information management apparatus according to another embodiment of the present invention.

FIG. 8 illustrates an overview of an auscultation system of another embodiment of the present invention.

FIG. 9 is a block diagram illustrating the hardware configuration of an information management apparatus according to another embodiment of the present invention.

FIG. 10 illustrates examples of live view images of a patient captured by an imaging unit of the information management apparatus.

FIG. 11 illustrates a specific example of a data structure of an auscultation-assisting-information database stored in an auscultation-assisting-information storage section of the information management apparatus.

FIG. 12 illustrates specific examples of auscultation assisting information stored in the auscultation-assisting-information storage section of the information management apparatus.

FIG. 13 illustrates a specific example of a projection image generated by an output control section of the information management apparatus.

FIG. 14 illustrates a usage mode in which a projection image is projected on the actual body of a patient.

FIG. 15 illustrates usage modes in which a projection image is projected on the actual body of a patient.

FIG. 16 illustrates a usage mode in which a projection image is projected on the actual body of a patient.

FIG. 17 illustrates a specific example of measurement position information and body sound information stored in a body-sound-information storage section of the information management apparatus.

FIG. 18 is a flowchart illustrating a flow of information management processing performed by the information management apparatus.

FIG. 19 illustrates an example in which a screen for instructing an operator to change a capturing range is displayed in a display unit of the information management apparatus.

FIG. 20 illustrates another specific example of a projection image generated by the output control section of the information management apparatus.

FIG. 21 illustrates an outer appearance of a digital stethoscope of still another embodiment of the present invention.

FIG. 22 is a functional block diagram illustrating the configuration of the major parts of an information management apparatus according to still another embodiment of the present invention.

FIG. 23 illustrates a specific example of a patient image of a patient which is being subjected to auscultation obtained by an imaging unit of the information management apparatus.

FIG. 24 illustrates a specific example of a model image indicating a measurement position generated by a measurement position information generator of the information management apparatus.

FIG. 25 is a block diagram illustrating an overview of a measurement system and the major parts of the configuration of an imaging apparatus forming the measurement system.

### DESCRIPTION OF EMBODIMENTS

### First Embodiment

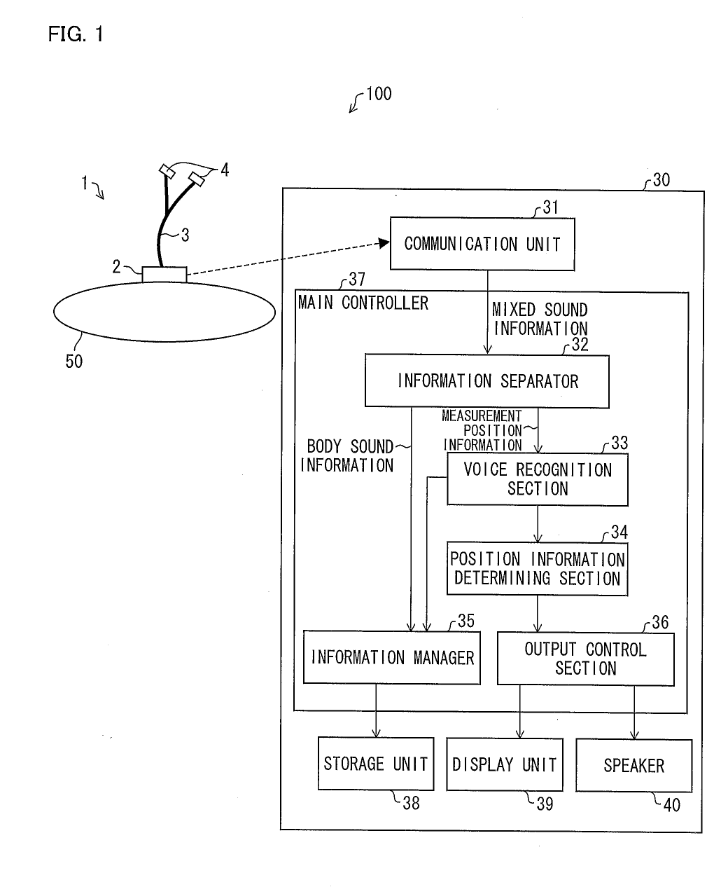

An embodiment of the present invention will be described below with reference to FIGS. 1 through 6.

### (Overview of Body Sound Measurement System 100)

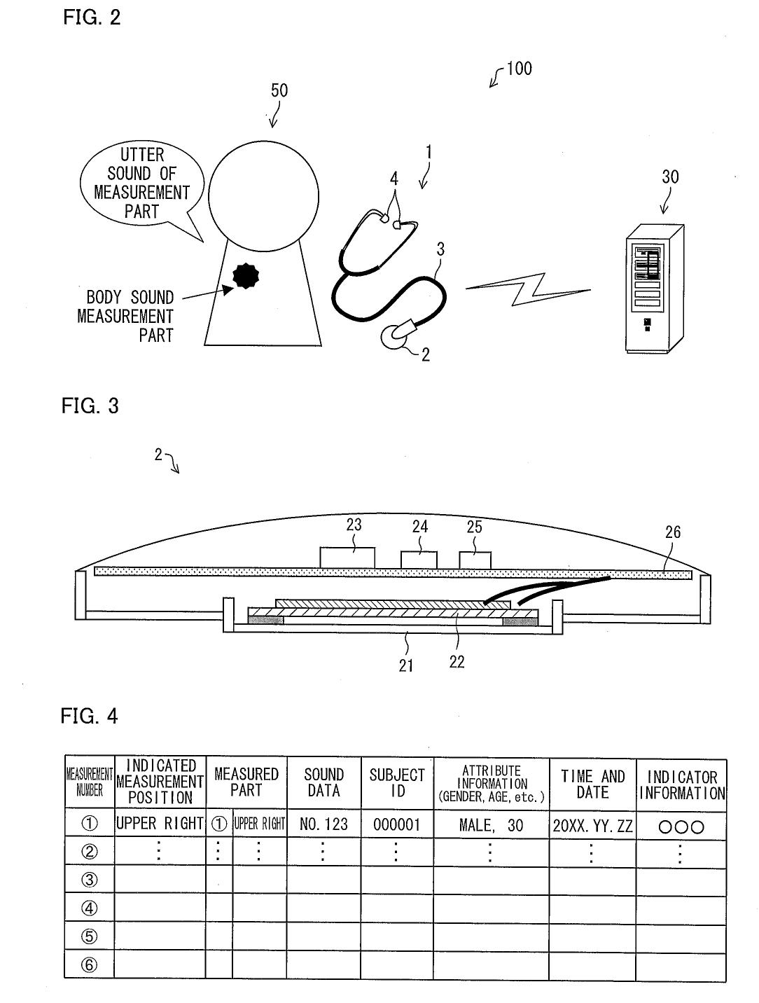

An overview of a body sound measurement system (information management system) 100 of an embodiment of the present invention will first be described below with reference to FIG. 2.

FIG. 2 illustrates an overview of the body sound measurement system 100. The body sound measurement system 100 includes, as shown in FIG. 2, a stethoscope (digital stethoscope and sound collector) 1 and a terminal device (information management apparatus) 30.

By applying a chestpiece 2 of the stethoscope 1 to, for example, the chest of a subject (body) 50 to be measured, body sounds of the subject 50 can be obtained. The type of body sound is not particularly restricted, and heartbeats, breath sounds, or intestine sounds, for example, may be obtained. The obtained body sounds are sent to the terminal device 30 as body sound information and are managed in the terminal device 30.

When obtaining body sounds, a user (operator) of the stethoscope 1 utters a sound of a measurement position (position from which body sounds are obtained) or a measurement part (part from which body sounds are obtained) to which the chestpiece 2 has been applied, and performs voice input via the stethoscope 1. Measurement position information which has been input as voice information in this manner is sent to the terminal device 30. Then, the terminal device 30 records the body sound information and the measurement position information in association with each other.

The user of the stethoscope 1 is able to associate body sound information with position information merely by uttering a sound of a measurement position by the user.

In the present invention, the number of pairs of body sound information and position information may be one or may be plural. The number of pairs of body sound information and position information is not restricted as long as, concerning at least one item of body sound information, a position at which this item of body sound information has been obtained can be specified.

### (Configuration of Stethoscope 1)

FIG. 1 illustrates the configuration of the body sound measurement system 100. The stethoscope 1 includes, as shown in FIG. 1, the chestpiece 2, a cable 3, and an earphone 4.

The chestpiece 2 is a sound collecting unit which abuts on the surface of the body 50 so as to obtain body sounds emitted from the inside of the body. This chestpiece 2 also serves as a voice collecting unit for obtaining voice uttered by the user of the stethoscope 1.

Body sounds obtained by the chestpiece 2 are transmitted to the earphone 4 via the cable 3 as an electric signal, and the body sounds are converted into an acoustic signal by the earphone 4.

### (Chestpiece 2)

FIG. 3 is a sectional view illustrating an example of the configuration of the chestpiece 2. The chestpiece 2 includes, as shown in FIG. 3, a diaphragm face (first sound collecting unit) 21, a vibration sensor (first sound collecting unit) 22, a microphone (second sound collecting unit) 23, a digital signal converter 24, and a communication unit (transmitter) 25. The microphone 23, the digital signal converter 24, and the communication unit 25 are disposed on a substrate 26.

Although the chestpiece 2 includes a battery for supplying power to the individual elements, it is not shown since it is not related to the features of the present invention.

### (Diaphragm Face 21)

When the subject 50 emits body sounds by, for example, breathing, the diaphragm face 21 performs micro-vibration in accordance with the waveform of the body sounds. The micro-vibration of the diaphragm face 21 is transmitted to the vibration sensor 22 via air.

### (Vibration Sensor 22)

The vibration sensor 22 detects micro-vibration of the diaphragm face 21 and converts the detected micro-vibration into an electric signal. In the example shown in FIG. 3, a sensor including a piezoelectric vibration sheet is used as the vibration sensor 22. This piezoelectric vibration sensor is constituted by two layers, that is, piezoelectric ceramics (upper layer) and a metallic sheet (lower layer). More specifically, the piezoelectric ceramics are sandwiched between two electrodes, and these electrodes are not shown in FIG. 3. The vibration sensor 22 is not restricted to the configuration shown in FIG. 3.

The vibration sensor 22 may include a filter (low-pass filter) for attenuating high frequency sounds (for example, sounds exceeding 1 kHz). Most body sounds have frequencies at 1 kHz or lower. Thus, by attenuating sounds exceeding 1 kHz, body sounds with reduced noise can be obtained.

An electric signal generated by the vibration sensor 22 is transmitted to the earphone 4 via the cable 3 and is also output to the digital signal converter 24.

In this embodiment, the configuration in which the voice of a user is detected by the vibration sensor 22 is also assumed. Since voice has usually frequencies exceeding 1 kHz, part of the voice is attenuated by the above-described low-pass filter. However, the voice is not completely attenuated, and thus, it is still possible to obtain the voice.

Alternatively, the above-described low-pass filter may not be disposed so that voice can be prevented from being attenuated.

### (Microphone 23)

The microphone 23 is a sound collector specially used for obtaining the voice of a user as measurement position information. The voice of a user may be obtained by using the diaphragm face 21 and the vibration sensor 22 as described above. Alternatively, in order to more reliably obtain the voice of a user, the microphone 23 may be disposed separately from the vibration sensor 22.

If the voice of a user and body sounds are obtained by the vibration sensor 22 and so on, it is preferable that voice and body sounds are separated in order to perform voice recognition, which will be discussed later. On the other hand, if voice is obtained by the microphone 23, only voice can be obtained, so that it is not necessary to separate body sounds from the voice for the purpose of improving the voice analyzing precision.

Additionally, if voice is obtained by using the vibration sensor 22 and so on, voice which has propagated within a body is obtained. However, if the microphone 23 is used, voice which has propagated in air can be obtained. Accordingly, clearer voice can be obtained by using the microphone 23 than by using the vibration sensor 22.

### (Digital Signal Converter 24)

The digital signal converter 24 converts an electric signal converted by the vibration sensor 22 into a digital signal and outputs the digital signal to the communication unit 25 as body sound information and measurement position information.

If the voice of a user (that is, measurement position information) is obtained by using the microphone 23, the digital signal converter 24 also converts an electric signal output from the microphone 23 into a digital signal and outputs the digital signal to the communication unit 25 as measurement position information.

The digital signal converter 24 may convert body sound information and measurement position information into sound data of a predetermined file format. Examples of the file format are MP3, WAV, MMA, MP2, AC3, OGG, RA, and AAC.

### (Communication Unit 25)

The communication unit 25 serves as a transmitter that transmits body sound information and measurement position information output from the digital signal converter 24 to a communication unit 31 of the terminal device 30.

The communication unit 25 may also serve the function of a receiver so that conditions concerning settings for the stethoscope 1 can be changed via the terminal device 30.

### (Configuration of Terminal Device 30)

The terminal device 30 is a device that manages body sound information and measurement position information obtained by the stethoscope 1. The terminal device 30 is, for example, a personal computer, a smartphone, or a PDA (personal digital assistant), though it is not particularly restricted.

The terminal device 30 includes, as shown in FIG. 1, the communication unit 31, a main controller (obtaining means) 37, a storage unit (memory unit) 38, a display unit (informing unit) 39, and a speaker (informing unit) 40.

The communication unit 31 sends and receives information to and from the stethoscope 1, and particularly serves as a receiver that receives body sound information and measurement position information from the stethoscope 1.

The storage unit 38 records programs executed by the main controller 37, such as (1) a control program for the individual elements, (2) an OS program, and (3) application programs, and also records (4) various items of data which are read when executing these programs. The storage unit 38 is constituted by a non-volatile storage device, such as a hard disk or a flash memory. In particular, the storage unit 38 stores therein body sound information and measurement position information received by the communication unit 31.

The display unit 39 is, for example, a liquid crystal display, and displays a screen for managing body sound information and measurement position information, a screen for indicating a predetermined measurement position to a user of the stethoscope 1, a screen for indicating an error message to the user of the stethoscope 1, and so on.

The speaker 40 outputs warning sounds for informing the user of the stethoscope 1 that a measurement position is not correct.

### (Main Controller 37)

The main controller 37 controls the terminal device 30, and particularly serves as obtaining means for obtaining body sound information and measurement position information. The main controller 37 manages body sound information and measurement position information in association with each other, and also determines whether or not a measurement position indicated by the voice uttered by the user matches a predetermined measurement position.

The main controller 37 includes, as shown in FIG. 1, an information separator (extracting means) 32, a voice recognition section (voice recognition means) 33, a position information determining section (determining means) 34, an information manager (associating means) 35, and an output control section (informing control means) 36.

### (Information Separator 32)

If the communication unit 31 receives measurement position information together with body sound information as the same sound data, the information separator 32 separates sounds included in the sound data into voice as measurement position information and sounds as body sound information. In other words, the information separator 32 independently extracts measurement position information and body sound information from the sound data. In this case, the information separator 32 may be regarded as obtaining means for obtaining measurement position information and body sound information.

The information separator 32 performs, for example, fast Fourier transform (FFT) on sound data (mixed sound information) indicating both of voice, which serves as measurement position information, and body sounds so as to separate sounds (body sounds) having frequencies at 1 kHz or lower and sounds (voice) having frequencies higher than 1 kHz. That is, the information separator 32 divides information concerning sounds contained in the sound data into two portions by using a predetermined frequency as a boundary. Then, the information separator 32 generates a body sound data file concerning body sounds and a voice data file concerning voice. The format of the data files may be one of the above-described formats, and is not particularly restricted. Voice information indicates voice expressing a measurement position, and may thus be considered as measurement position information.

The body sound data file generated by the information separator 32 is output to the information manager 35, while the voice data file (that is, measurement position information) generated by the information separator 32 is output to the voice recognition section 33.

In order to clarify the association between the body sound data file and the voice data file, the information separator 32 may provide a file name indicating that the body sound data file and the voice data file are associated with each other to the body sound data file and the voice data file generated from the same sound data file.

### (Voice Recognition Section 33)

The voice recognition section 33 performs voice recognition on voice (measurement position information) indicated by the voice data file generated by the information separator 32. More specifically, the voice recognition section 33 analyzes voice indicated by the voice data file so as to convert measurement position information expressed by voice into characters or numbers or a combination thereof (hereinafter referred to as “characters and so on”). The voice recognition section 33 then outputs measurement position information expressed by characters and so on to the information manager 35 and the position information determining section 34.

A voice recognition method performed by the voice recognition section 33 is not particularly restricted. For example, the voice recognition section 33 may recognize all words and phrases contained in voice or may recognize only predetermined words and phrases contained in voice.

### (Position Information Determining Section 34)

The position information determining section 34 compares a measurement position indicated by the measurement position information output from the voice recognition section 33 with a measurement position which is being specified at this time point. If two items of information do not match each other, the position information determining section 34 outputs disparity information indicating that the two items of information do not match each other to the output control section 36.

It is possible that a user of the stethoscope 1 may incorrectly recognize a predetermined measurement position and utter a sound of the recognized measurement position. For example, if a measurement position is indicated in an image which schematically indicates the chest of a subject, it is possible that the user may misrecognize that the indicated position is on a right side as viewed from the user or as viewed from the subject.

Even in this case, by providing the position information determining section 34, it is possible to reduce the possibility that measurement will continue after the user has misrecognized a predetermined measurement position.

### (Output Control Section 36)

The output control section 36 controls the display unit 39 and the speaker 40. In particular, upon receiving disparity information from the position information determining section 34, the output control section 36 displays a message indicating that a measurement position is not correct in the display unit 39 and also outputs warning sounds from the speaker 40.

### (Information Manager 35)

The information manager 35 manages body sound information and measurement position information separated by the information separator 32 in association with each other.

More specifically, the information manager 35 creates a table (association table) for associating an identifier (for example, a file name) of body sound information with an identifier (for example, a file name) of measurement position information indicated as voice information, and stores the association table in the storage unit 38.

Alternatively, the information manager 35 may associate an identifier of body sound information with measurement position information indicated as characters or numbers obtained by performing voice recognition by the voice recognition section 33.

FIG. 4 illustrates an example of the association table. In the example shown in FIG. 4, a predetermined measurement position (such as upper right), an actually measured position (indicated by “measured part” in FIG. 4) (such as upper right), an identifier (such as No. 123) of sound data (body sound information), an identifier of a subject, and so on, are associated with each other. Since the number of subjects may be only one, it is sufficient that at least body sound information and measurement position information are associated with each other in the association table.

In the configuration in which body sound information is obtained by the vibration sensor 22 and so on, and voice information is obtained by the microphone 23, voice information output from the microphone 23 can be handled as measurement position information. Accordingly, it is not always necessary to provide the information separator 32. In this case, voice information output from the microphone 23 can be directly input into the voice recognition section 33 without using the information separator 32. Body sound information may also be directly input into the information manager 35 without using the information separator 32.

In this configuration, the information manager 35 may be regarded as obtaining means for obtaining body sound information, while the voice recognition section 33 may be regarded as obtaining means for obtaining measurement position information.

### (Flow of Processing Performed by Body Sound Measurement System 100)

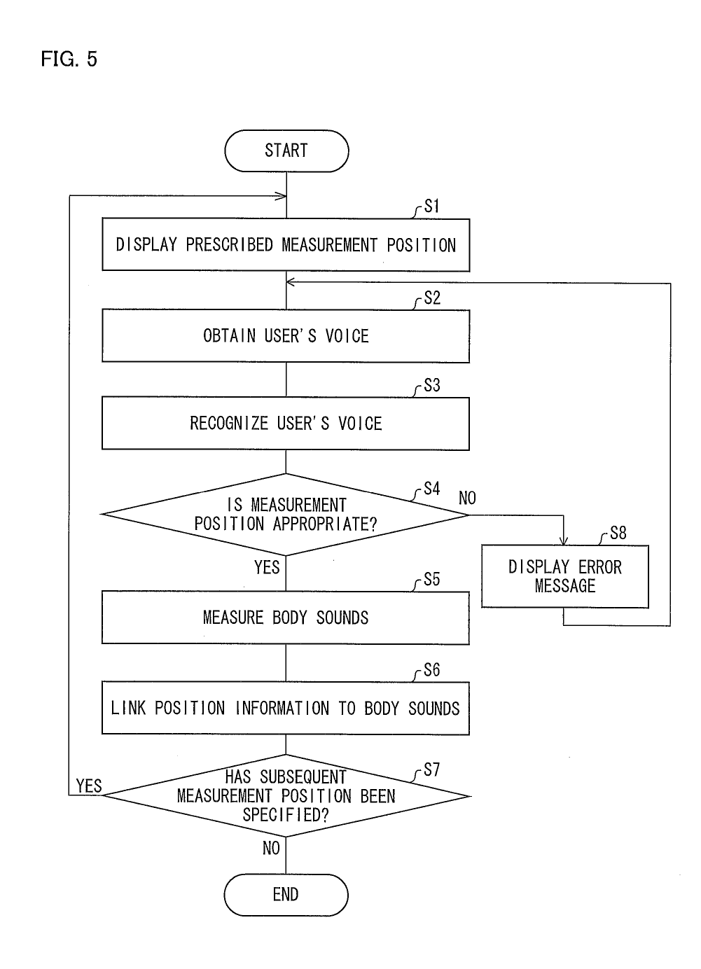

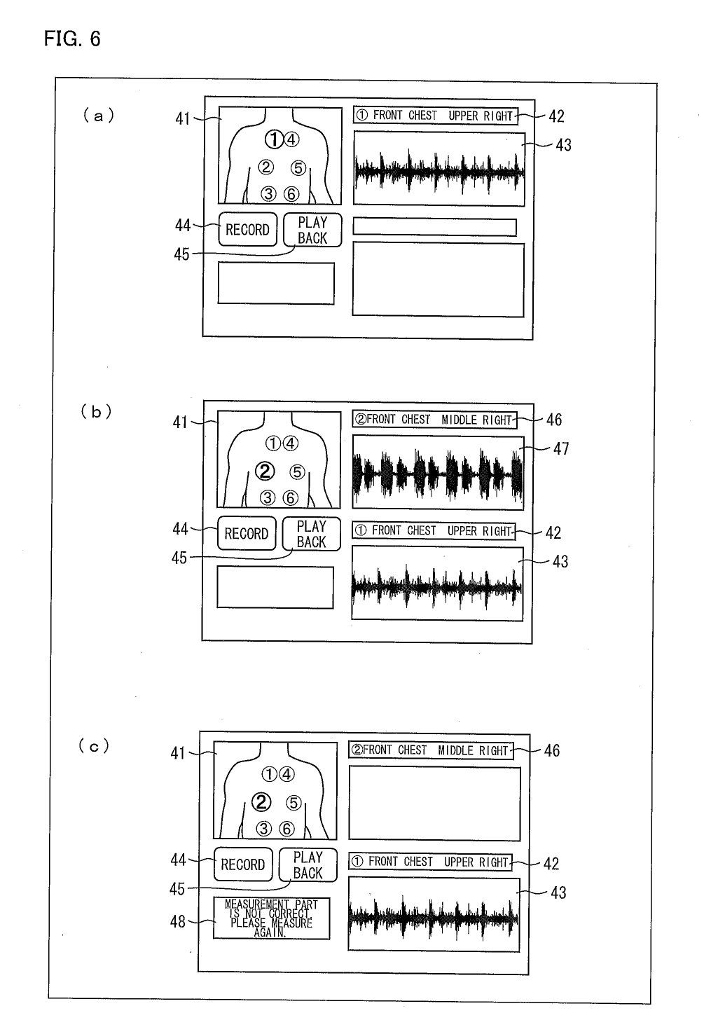

An example of a flow of processing performed by the body sound measurement system 100 will be described with reference to FIGS. 5 and 6. FIG. 5 is a flowchart illustrating an example of a flow of processing performed by the body sound measurement system 100. FIG. 6 illustrates examples of screens displayed in the display unit 39. In this example, a description will be given of a flow of processing executed when body sounds are measured after a user of the stethoscope 1 has uttered a sound of a measurement position.

First, a prescribed measurement position is displayed in the display unit 39 (S1). Concerning which measurement position will be selected at a certain time point, the main controller 37 makes a determination on the basis of information concerning measurement positions stored in the storage unit 38. The output control section 36 performs display control for a measurement position in accordance with a determination made by the main controller 37. The measurement order is determined for a plurality of measurement positions, and measurement positions are selected in accordance with this predetermined order.

In the example shown in part (a) of FIG. 6, in an image 41, first through sixth measurement positions are indicated on a schematic representation of a body image, and, among these measurement positions, a first measurement position is highlighted. A description of the first measurement position (“front chest, upper right”) is displayed as an image 42.

Then, the user of the stethoscope 1 utters sounds of information (measurement position information) for specifying the first measurement position toward the chestpiece 2. For example, the user of the stethoscope 1 utters “upper right” and “the first”. These sounds are converted into an electric signal by the vibration sensor 22 and the electric signal is then converted into a digital signal by the digital signal converter 24. The digital signal is then transmitted from the communication unit 25 to the communication unit 31 of the terminal device 30 as voice information. Alternatively, these sounds are obtained by the microphone 23 and are converted into a digital signal by the digital signal converter 24. The digital signal is then transmitted from the communication unit 25 to the communication unit 31 as voice information.

The voice information received by the communication unit 31 is input into the main controller 37 (first obtaining step) (S2), and is then output to the voice recognition section 33. Then, the voice recognition section 33 recognizes voice indicated by the voice information (S3). In this example, since measurement position information is obtained prior to body sound information, it is not always necessary to provide the information separator 32.

If the sound of a measurement position is uttered while auscultation is being performed, body sound information and measurement position information are obtained at the same time (as one piece of sound data). Accordingly, it is preferable that the body sound information and the measurement position are separated. In this case, mixed sound information indicating both of the body sound information and the measurement position information is input into the information separator 32. As discussed above, the information separator 32 separates the mixed sound information into the body sound information and the measurement position information, and outputs the body sound information to the information manager 35 and the measurement position information to the voice recognition section 33.

The voice recognition section 33 outputs voice recognition results (measurement position information in the form of characters or numbers) to the information manager 35 and the position information determining section 34.

The position information determining section 34 determines whether or not a measurement position indicated by the obtained measurement position information matches a measurement position which is currently selected (S4).

If the position information determining section 34 has determined that the two items of information match each other (YES in S4), the measurement and recording of body sounds is started when a record button 44 is pressed by the user (S5). Body sound information obtained by the stethoscope 1 is sent to the communication unit 31 and is input into the main controller 37 (second obtaining step). The body sound information is then output to the information manager 35 directly or via the information separator 32.

Upon receiving body sound information, the information manager 35 creates an association table for associating the obtained measurement position information with the body sound information, and stores the created association table, together with the body sound information and the measurement position information, in the storage unit 38 (S6) (associating step).

The output control section 36 displays a waveform of the measured body sounds in the display unit 39 as an image 43.

Then, the main controller 37 determines whether or not a subsequent measurement position has been specified, on the basis of information concerning measurement positions stored in the storage unit 38 (S7).

If the subsequent measurement position has been specified (YES in S7), the output control section 36 highlights a second measurement position in the image 41, as shown in part (b) of FIG. 6, and displays a description of the second measurement position (“front chest, middle right”) as an image 46.

When body sound information concerning the second measurement position is obtained, the output control section 36 displays a waveform of the second body sounds in the display unit 39 as an image 47.

If the position information determining section 43 has determined that the measurement position indicated by the measurement position information does not match the measurement position which is currently selected (NO in S4), the position information determining section 34 outputs disparity information indicating that the two items of information do not match each other to the output control section 36.

Upon receiving disparity information, the output control section 36 displays a message 48 indicating the measurement position is not correct in the display unit 39, as shown in part (c) of FIG. 6, and also outputs warning sounds from the speaker 40 (S8).

Then, the main controller 37 waits until a sound of measurement position information indicating a correct measurement position is input (returns to S2).

If it is determined that measurement at all of the predetermined measurement positions has finished (NO in S7), the main controller 37 terminates the entire processing.

A playback button 45 is a button for playing back body sound information stored in the storage unit 38.

### Variation Examples

### Example in which the Obtaining Order of Body Sound Information and Measurement Position Information is Changed

The terminal device 30 may obtain body sound information first and then obtain measurement position information. In this case, in order to clarify the association between body sound information and measurement position information, measurement position information obtained within a predetermined period after body sound information has been obtained by the stethoscope 1 is regarded as measurement position information associated with this body sound information obtained first. A time point at which body sound information or measurement position information is obtained by the stethoscope 1 is a time point at which the body sound information or the measurement position information is buffered in a temporary storage memory (storage unit) of the stethoscope 1.

Alternatively, measurement position information sent (or received) within a predetermined period after body sound information has been sent by the communication unit 25 (or received by the communication unit 31) may be regarded as measurement position information associated with this body sound information obtained first.

In order to implement such a configuration, the information manager 35 manages times at which body sound information and measurement position information are obtained (or sent or received), and determines the association between body sound information and measurement position information on the basis of the relationship between a time at which the body sound information has been obtained (or sent or received) and a time at which the measurement position information has been obtained (or sent or received). For implementing the management and determination performed by the information manager 35, time information indicating a time at which body sound information has been obtained (or sent or received) is added to the body sound information, and time information indicating a time at which measurement position information has been obtained (or sent or received) is added to the measurement position information. Alternatively, the association between body sound information and measurement position information and time information thereof may be indicated by, for example, a table.

This variation may be applied to a case in which measurement position information is obtained first and then body sound information is obtained. That is, body sound information obtained within a predetermined period after measurement position information has been obtained by the stethoscope 1 is regarded as body sound information associated with this measurement position information obtained first. Alternatively, body sound information sent (or received) within a predetermined period after measurement position information has been sent by the communication unit 25 (or received by the communication unit 31) is regarded as body sound information associated with this measurement position information obtained first.

In this manner, if body sound information and measurement position information are sent and received as different files, it is preferable that the association between body sound information and measurement position information is determined by using a certain technique.

In contrast, if body sound information and measurement position information are sent and received as the same file, the association between the body sound information and measurement position information is clear, and it is not necessary to determine the association therebetween when a sound file is received. From this point of view, it is preferable that body sound information and measurement position information are sent and received as the same file. Accordingly, voice (measurement position information) obtained by the microphone 23 and body sounds obtained by the vibration sensor 22 may be first included in one sound data file, and then, the sound data file may be sent to the communication unit 31.

(Stethoscope 1 without Communication Unit 25)

It is not always necessary that the stethoscope 1 include the communication unit 25. The stethoscope 1 may include a non-volatile storage unit that is capable of storing body sound information and measurement position information, so that data can be transferred from the storage unit of the stethoscope 1 to the storage unit 38 of the terminal device 30. In this case, the main controller 37 obtains body sound information and measurement position information from the storage unit 38.

In this configuration, it is preferable that the stethoscope 1 at least has a function corresponding to the information manager 35 so as to associate body sound information with measurement position information.

Alternatively, the stethoscope 1 may have a function similar to the main controller 37.

(Configuration in which Noise Canceling is Performed)

If body sound information and measurement position information are separately obtained (they are not obtained at the same time), noise picked by the microphone 23 may be used for canceling noise included in body sounds obtained by the vibration sensor 22 and so on. That is, sounds in an opposite phase of noise picked by the microphone 23 may be generated by a digital circuit or an analog circuit, and the generated signal is superposed on a body sound signal, thereby attenuating noise included in the body sounds. The stethoscope 1 may include such a circuit configuration.

(Example in which the Configuration of Terminal Device 30 is Changed)

The terminal device 30 may be implemented as a server. In this case, it is preferable that a second terminal device that is possible to dispose near a user of the stethoscope 1 is prepared, and that the output control section 36, the display unit 39, and the speaker 40 are included in the second terminal device. Information for specifying a measurement position and the above-described disparity information are sent from the terminal device 30, which serves as a server, to the second terminal device.

### (Advantages of Body Sound Measurement System 100)

In the body sound measurement system 100, a user is able to input measurement position information via the stethoscope 1. Accordingly, it is not necessary to provide an interface for inputting characters, such as a keyboard, and body sound information and measurement position information can be associated with each other merely by using the stethoscope. Such a configuration is especially useful when a physician, for example, is dispatched to a remote site where sufficient equipment is not provided.

The stethoscope is a sound collector by itself. Accordingly, it is not always necessary to separately provide a sound input device for inputting measurement position information. Thus, measurement position information can be obtained with a simple configuration.

In particular, in the configuration in which body sound information and measurement position information are obtained as the same sound data, the association between body sound information and measurement position information is clear. Thus, body sound information and measurement position information can be easily managed in association with each other.

### Second Embodiment

Another embodiment of the present invention will be described below.

### Background Art

Hitherto, digital stethoscopes which collect body sounds (such as breath sounds and heartbeats) from a body (patient or subject) and record the collected body sounds as digital signals (body sound information) are widely used. By digitally recording body sound information by using a digital stethoscope, a great variety of modes of diagnosis are implemented, which are different from existing modes, for example, a physician examines a patient on a face-to-face basis by using a stethoscope. For example, a physician being in a place away from a patient and an operator of a digital stethoscope is able to receive information concerning collected body sounds and conduct diagnosis in a remote site. Additionally, the use of a digital stethoscope makes it possible for a physician to listen to collected and recorded body sound information later, so that the physician can compare items of information concerning body sounds collected on different dates with each other.

Various techniques concerning digital stethoscopes for implementing the above-described diagnosis modes are available.

PTL 1 discloses the following auscultation system. While an image of appropriate-point marks indicating appropriate positions and a recording order thereof is being displayed on a simulated patient image, a nurse, for example, is instructed to collect body sounds by using a stethoscope. In this auscultation system, an auscultation sound signal indicating obtained auscultation sounds is recorded as one file, and appropriate-point identification data indicating a preset appropriate point on a body surface of a patient is added to this file.

PTL 3 discloses the following telemedicine diagnosis system. Symbols of standard diagnostic positions to which a stethoscope is supposed to be applied are superposed on an image captured from a body of a patient or on simply represented graphics of the body of the patient, so that a user can be informed of the standard diagnostic positions through a medical service window.

PTL 4 discloses the following diagnosis system. A measurement position at which a stethoscope is being applied is recognized on the basis of an image captured by a digital camera, and an operator performing this image capturing operation is instructed to check whether or not information concerning a recognized measurement position is correct. In this diagnosis system, auscultation sounds are collected when a recognized measurement position is correct, thereby automatically obtaining measurement position information.

### Summary of Invention

### Technical Problem

When obtaining body sounds of a patient by using a digital stethoscope, measurement position information indicating a portion of a body surface of a patient from which body sounds have been obtained by applying the digital stethoscope to this portion is important. Body sounds are location-dependent, and obtained body sounds are different depending on where a stethoscope is applied. As discussed above, in a diagnosis mode in which a physician is in a remote site or a physician listens to body sound information on a date other than a date at which the body sound information has been collected, it is particularly important to manage body sound information together with measurement position information. That is, unless body sound information of a patient obtained by a digital stethoscope is correlated with a measurement position of the patient from which the body sound information has been obtained, such information is meaningless.

In the system disclosed in PTL 1, for linking predetermined appropriate-point identification data to one file, an operator of a digital stethoscope has to perform auscultation by remembering rules determined for each appropriate point. Additionally, auscultation sounds continuously collected from when the recording of such auscultation sounds is started until it is finished by using an ON/OFF switch are handled as one file, and every time one file is recorded, the operator has to specify information concerning a measurement position. In this manner, in the system disclosed in PTL 1, a recording operation for auscultation sounds becomes complicated for the operator.

PTL 3 does not disclose a configuration in which body sound information and measurement position information are associated with each other.

In the system disclosed in PTL 4, a special medical digital camera including dedicated spectacle lenses is required, and an operator (for example, a physician) has to perform all operations, such as image capturing of the progress of auscultation by operating this medical digital camera, checking whether or not a measurement position is suitable, and performing auscultation. In this system, as discussed above, a special device is required, and also, an operation is so burdensome that an operator is not able to concentrate on auscultation.

The present invention has been made in view of the above-described problems. It is an object of the present invention to provide an information management apparatus that allows an operator to perform measurement by using a digital stethoscope without using a special device involving a complicated operation and that makes it easy to link collected body sound information to measurement position information at which the body sound information has been obtained, and also to provide an information management method, a control program, and a recording medium.

### Description of Embodiments

### First Mode of Second Embodiment

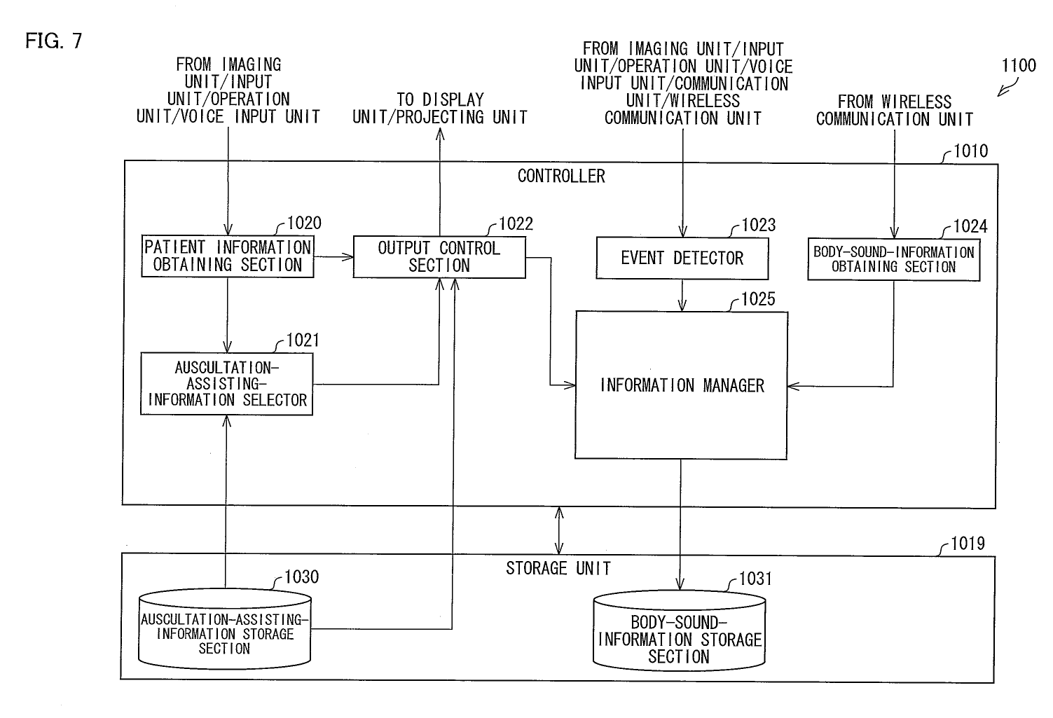

An embodiment of an information management apparatus of the present invention will be described below with reference to FIGS. 7 through 21.

In the following embodiment, an example in which an information management apparatus of the present invention is applied to an auscultation system will be discussed. The auscultation system is, in this example, a system that implements the following operation. Body sounds of a subject are obtained by using a digital stethoscope, and obtained digital data, that is, body sound information, is managed by the information management apparatus of the present invention and is used for medical diagnosis and treatment for the subject. A subject to be subjected to a medical examination by using a digital stethoscope will be referred to as a “patient”. Although in this embodiment a human being is assumed as a subject (patient), an auscultation system in which all sorts of living bodies other than human beings are assumed as subjects (patients) is also encompassed within the present invention.

The information management apparatus of the present invention is not restricted to the system in the above-described example, and may be applied to all sorts of other systems in which body sound information is obtained from a living body and is utilized for a purpose other than medical diagnosis and treatment.

### [Overview of Auscultation System]

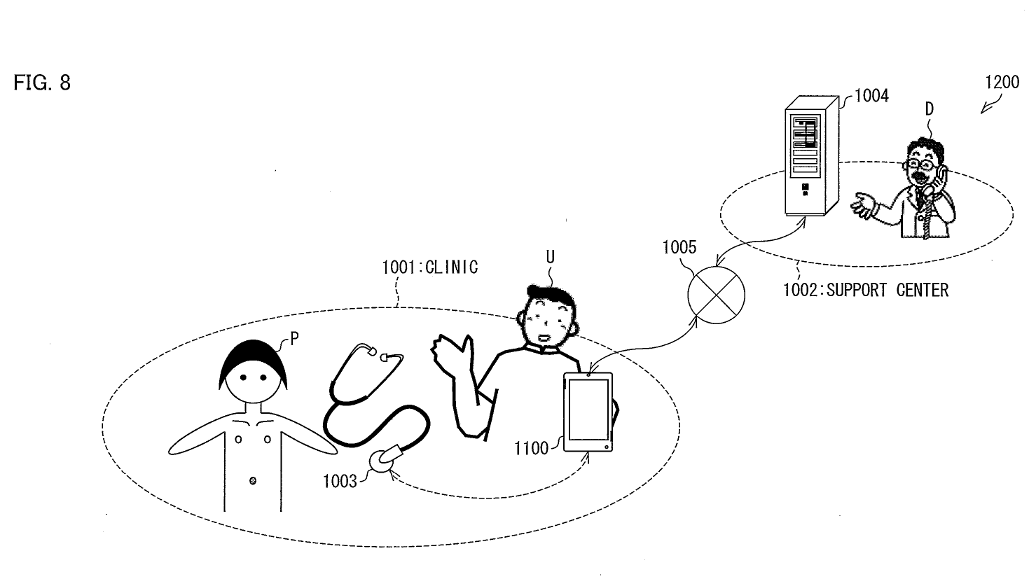

FIG. 8 illustrates an overview of an auscultation system of an embodiment of the present invention. An auscultation system 1200 at least includes a digital stethoscope 1003 used for collecting (that is, auscultating) body sounds from a patient P by an operator U, and an information management apparatus 1100 used by the operator U when auscultating body sounds.

The operator U is in a clinic 1001 where medical diagnosis and treatment is given to the patient P, and examines the patient P in the clinic 1001 by using various devices, such as the digital stethoscope 1003. In this case, the various devices may include an oximeter, an electrocardiograph, a sphygmomanometer, a thermometer, an arteriosclerosis meter, and a blood vessel aging measuring device.

The information management apparatus 1100 and the digital stethoscope 1003 are connected to each other so that they can communicate with each other via a wired or wireless medium. By operating the information management apparatus 1100, the operator U is able to read and refer to information necessary to examine the patient P, for example, information concerning the patient P or a diagnosis procedure. The operator U is also able to manage body sound information collected from the digital stethoscope 1003 in the information management apparatus 1100.

The information management apparatus 1100 is implemented by an information processing terminal having a high portability owned by the operator U, or a desk-top personal computer (PC) installed in the clinic 1001. In the example shown in FIG. 8, the information management apparatus 1100 of the present invention is implemented by a multifunction mobile communication terminal, such as a smartphone, by way of example.

If the operator U has medical expertise, skills, and authority as a physician, he/she may examine the patient P by using the digital stethoscope 1003 and the information management apparatus 1100, and may give treatment to the patient P by making a final judgment of the condition of the patient P. In this manner, an auscultation system including the digital stethoscope 1003 and the information management apparatus 1100 is also encompassed within the present invention.

The auscultation system 1200 shown in FIG. 8 is also encompassed within the present invention. That is, the auscultation system 1200 may be constructed by including the digital stethoscope 1003 and the information management apparatus 1100 in the clinic 1001 and also including a management server 1004 in a support center 1002 of a remote site. In this case, the information management apparatus 1100 and the management server 1004 are connected to each other so that they can communicate with each other via a communication network 1005, such as the Internet.

More specifically, the following situation may be considered. The operator U may have skills to operate the digital stethoscope 1003 and the information management apparatus 1100 and to perform simple medical checking and treatment on the spot in the clinic 1001 under the guidance of a specialized physician, though the operator U does not have the same levels of expertise, skills, and authority as those of the physician, or though the operator U is not a specialist of the field of currently conducted medical checking and treatment. Under this situation, the digital stethoscope 1003 and the information management apparatus 1100 operated by the operator U, such as a nurse practitioner (NP) or another health care professional, are disposed in the clinic 1001 of the auscultation system 1200, and in the support center 1002 located away from the clinic 1001, the management server 1004 which manages electronic health records of individual patients in the auscultation system 1200 is disposed. A physician D having special expertise and skills stays in the support center 1002, and gives guidance to the operator U by using a communication device (not shown), such as an information processing terminal or a telephone, so as to assist the operator U to conduct diagnosis and treatment. Meanwhile, body sound information directly collected from the patient P by the operator U by using the digital stethoscope 1003 is stored in the management server 1004 via the information management apparatus 1100. The physician D is able to give instructions concerning diagnosis and treatment by accessing the management server 1004 and by obtaining body sound information concerning the patient P being in a remote site. Under the guidance of the physician D, the operator U is able to conduct simple treatment, or if it is difficult to handle this patient P in the clinic 1001, the operator U is able to introduce a hospital, which may give a suitable treatment, cooperated with this clinic 1001.

As discussed above, it is necessary to manage body sound information collected from the digital stethoscope 1003, together with measurement position information indicating a measurement position of a body surface of the patient P at which the body sound information has been collected. Particularly, for enabling a physician D being in a remote site to suitably conduct diagnosis or to refer to electronic health records of the patient P on a date other than a date on which diagnosis has been conducted, it is essential that body sound information and measurement position information are linked to each other.

In this embodiment, the information management apparatus 1100 implemented by a smartphone links body sound information to measurement position information and suitably manages information concerning a patient P.

The configuration and the operation of this information management apparatus 1100 will be described below in detail.

### [Hardware Configuration of Information Management Apparatus]

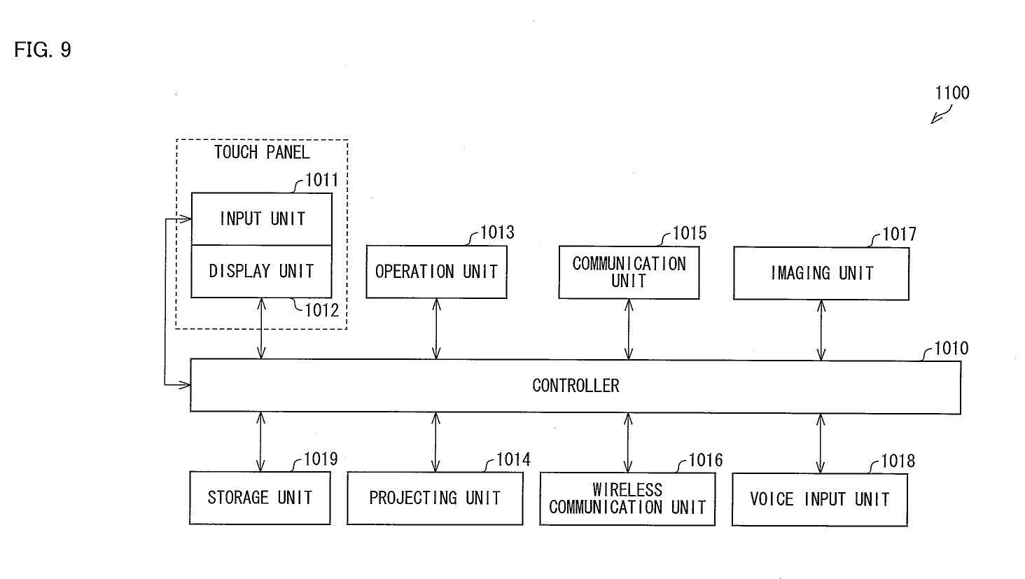

FIG. 9 is a block diagram illustrating the hardware configuration of the information management apparatus 1100 of this embodiment. The information management apparatus 1100 at least includes, as shown in FIG. 9, a controller 1010, an input unit 1011 or an operation unit 1013, a display unit 1012 or a projecting unit 1014, a wireless communication unit 1016, an imaging unit 1017, and a storage unit 1019. For implementing regular functions of a smartphone, the information management apparatus 1100 may also include a communication unit 1015 and a voice input unit 1018, and various regular components of a smartphone, such as an external interface, a sound output unit, a speech communication processor, a broadcasting receiver (such as a tuner and a demodulator), a GPS, and sensors (such as an acceleration sensor and an orientation sensor).

In this embodiment, since the information management apparatus 1100 is a smartphone, the input unit 1011 and the display unit 1012 are integrally formed as a touch panel. If the information management apparatus 1100 is implemented by, for example, a PC, the display unit 1012 may be implemented by, for example, a liquid crystal display monitor, and instead of the input unit 1011, the operation unit 1013 may be used and implemented by, for example, a keyboard and a mouse.

The input unit 1011 is used for allowing a user to input an instruction signal to operate the information management apparatus 1100 via the touch panel. The input unit 1011 is constituted by a touch face and a touch sensor. The touch face receives contact of a pointer (such as a finger or a pen). The touch sensor detects contact/non-contact (access/non-access) between a pointer and the touch face and also detects a contact (access) position. The touch sensor may be implemented by any type of sensor, for example, a pressure sensor, an electrostatic capacitive sensor, an optical sensor, as long as it is able to detect contact/non-contact between a pointer and the touch face.

The display unit 1012 displays information managed by the information management apparatus 1100 and also displays an operation screen for allowing a user to operate the information management apparatus 1100 as a GUI (Graphical User Interface) screen. The display unit 1012 is implemented by a display device, for example, an LCD (liquid crystal display).

The operation unit 1013 allows a user to directly input an instruction signal into the information management apparatus 1100. For example, the operation unit 1013 is implemented by a suitable input mechanism, such as a button, a switch, a key, or a jog dial. For example, the operation unit 1013 is a switch for turning ON/OFF the power of the information management apparatus 1100 or a dial for adjusting enlargement/reduction of a projected image output from the projecting unit 1014.

The projecting unit 1014 is a so-called projector, which receives a video signal processed and output from the controller 1010 and enlarges and projects the video signal as an optical image. The projector may be integrated in the information management apparatus 1100. Alternatively, the projector may be implemented as a device separately provided from the information management apparatus 1100. In this case, the projector is connected to the information management apparatus 1100 via a wired or wireless medium, and sends and receives a video signal to and from the information management apparatus 1100.

The communication unit 1015 communicates with external devices via a communication network. In this embodiment, the communication unit 1015 is connected to the management server 1004 (or an information terminal device of the physician D, which is not shown) in the support center 1002 via the communication network 1005 so that data can be sent and received between the information management apparatus 1100 and the management server 1004. If the information management apparatus 1100 is a cellular phone, such as a smartphone, the communication unit 1015 is able to send and receive voice communication data, email data, and so on, to and from other devices via a cellular phone circuit network.

The wireless communication unit 1016 wirelessly communicates with external devices. In this embodiment, the wireless communication unit 1016 performs wireless communication with the digital stethoscope 1003 so as to receive, from the digital stethoscope 1003, body sound information obtained by digitizing body sounds collected by the digital stethoscope 1003.

The type of wireless communication unit 1016 is not particularly restricted, and may implement one or a plurality of wireless communication means such as infrared communication, for example, IrDA or IrSS, Bluetooth (registered) communication, WiFi communication, and a non-contact IC card.

If the digital stethoscope 1003 and the information management apparatus 1100 communicate with each other via a wired medium, the wireless communication unit 1016 is not essential.

The imaging unit 1017 captures still images or moving images, and is constituted by a suitable imaging mechanism including lenses and imaging elements. For example, the imaging unit 1017 is implemented by a CCD (Charge Coupled Device) camera or a CMOS (Complementary Metal-Oxide-Semiconductor) camera. However, another imaging device may be used as the imaging unit 1017.

The voice input unit 1018 receives input of voice generated outside the information management apparatus 1100 and is implemented by, for example, a microphone. Voice input via the voice input unit 1018 may be subjected to voice recognition and may be converted into an instruction signal for the information management apparatus 1100.

The storage unit 1019 is a device that stores (1) a control program executed by the controller 1010 of the information management apparatus 1100, (2) an OS program executed by the controller 1010, (3) application programs for executing various functions of the information management apparatus 1100 by the controller 1010, and (4) various items of data which are read when these application programs are executed. Alternatively, the storage unit 1019 is a device that stores (5) data used for calculations while the controller 1010 is executing various functions and also stores calculation results. The above-described items of data (1) through (4) are stored in a non-volatile storage device, such as a ROM (read only memory), a flash memory, an EPROM (Erasable Programmable ROM), an EEPROM (registered trademark) (Electrically EPROM), or an HDD (Hard Disk Drive). The above-described item of data (5) is stored in a volatile storage device, such as a RAM (Random Access Memory). Decisions concerning which item of data will be stored in which storage device are suitably made by considering the purpose of use of the information management apparatus 1100, convenience, costs, physical restrictions. For example, body sound information concerning a patient P is temporarily stored in the storage unit 1019 implemented by a non-volatile storage device. Live view images captured by the imaging unit 1017 are temporarily stored in the storage unit 1019 implemented by a volatile storage device.

The controller 1010 centrally controls individual elements included in the information management apparatus 1100. The controller 1010 is implemented by, for example, a CPU (central processing unit). Functions of the information management apparatus 1100 are implemented by reading a program stored in, for example, a ROM, into, for example, a RAM, by a CPU used as the controller 1010. Various functions (in particular, an information management function) implemented by the controller 1010 will be discussed later in detail with reference to drawings different from FIG. 9.

### [Functional Configuration of Information Management Apparatus]

FIG. 7 is a functional block diagram illustrating the configuration of the major parts of the information management apparatus 1100 of this embodiment.

As shown in FIG. 7, the controller 1010 of the information management apparatus 1100 includes, as functional blocks, a patient information obtaining section 1020, an auscultation-assisting-information selector 1021, an output control section 1022, an event detector 1023, a body-sound-information obtaining section 1024, and an information manager 1025.

The above-described functional blocks of the controller 1010 are implemented as a result of, for example, a CPU (central processing unit), reading a program stored in a storage device (storage unit 1019) implemented by, for example, a ROM (read only memory) or an NVRAM (non-volatile random access memory), into, for example, a RAM (random access memory), and executing the read program.

The storage unit 1019 is a storage unit from and to which data is read or written when the above-described elements of the controller 1010 execute the information management function of the auscultation system. More specifically, the storage unit 1019 at least includes an auscultation-assisting-information storage section 1030. The storage unit 1019 may also include a body-sound-information storage section 1031.

The patient information obtaining section 1020 obtains all items of information concerning a patient which are input into the information management apparatus 1100. All items of information concerning a patient (hereinafter simply referred to as “patient information”) include at least one of still images and moving images of the patient captured by the capturing unit 1017, patient information input via the input unit 1011, patient information input via the operation unit 1013, and voice uttered for patient information or voice of the patient himself/herself input via the voice input unit 1018.

Patient information (subject information) obtained by the patient information obtaining section 1020 is supplied to the auscultation-assisting-information selector 1021. The auscultation-assisting-information selector 1021 refers to the patient information in order to select an optimal item of auscultation assisting information.

The auscultation-assisting-information selector 1021 selects an item of auscultation assisting information suitable for a patient, on the basis of the above-described patient information. The auscultation assisting information is information referred to by an operator U when conducting auscultation, and at least includes measurement position information (information indicating a position on a body surface of a patient to which a stethoscope is supposed to be applied) so that the operator U can conduct suitable auscultation for the patient. The auscultation assisting information may also include various items of information useful for the operator U to conduct auscultation, such as an auscultation procedure and cautions in conducting auscultation. Optimal auscultation may vary depending on a patient. In particular, measurement position information may vary depending on patient attributes (particularly, body-build). When the above-described patient information is obtained, the information management apparatus 1100 identifies patient attributes on the basis of the patient information. The auscultation-assisting-information selector 1021 selects, in accordance with the patient attributes, an item of auscultation assisting information indicating useful information so as to conduct optimal auscultation for the patient. For example, the auscultation-assisting-information selector 1021 may select auscultation assisting information indicating measurement position information suitable for the body-build of the patient. Several patterns of the auscultation assisting information are stored in accordance with assumed patient attributes in the auscultation-assisting-information storage section 1030 in advance.

The output control section 1022 converts an item of auscultation assisting information selected by the auscultation-assisting-information selector 1021 into a video signal and outputs the video signal to a video signal output unit. The video signal output unit is a suitable output device that can process a video signal and display the processed video signal so that the operator U can visually check it. For example, the display unit 1012 and the projecting unit 1014 are included in the video signal output unit. That is, the output control section 1022 is able to output auscultation assisting information to the display unit 1012 and causes the display unit 1012 to display it, or to output auscultation assisting information to the projecting unit 1014 and causes the projecting unit 1014 to project it on, for example, a screen. The operator U refers to auscultation assisting information displayed in the display unit 1012 or on a screen so as to conduct auscultation suitable for the patient.

The event detector 1023 monitors the individual elements of the information management apparatus 1100 and detects an event occurring in the information management apparatus 1100. In this embodiment, when detecting an event satisfying a specific condition, the event detector 1023 informs the information manager 1025 of the occurrence of this event. One type of event to be detected by the event detector 1023 is an event which serves as a trigger for causing the information manager 1025 to store body sound information in the body-sound-information storage section 1031. For example, examples of such events detected by the event detector 1023 may be capturing of a specific image by the imaging unit 1017, inputting of a specific instruction signal via the input unit 1011, inputting of a specific instruction signal via the operation unit 1013, inputting of a specific voice signal via the voice input unit 1018, inputting of specific information via the communication unit 1015, and inputting of specific information via the wireless communication unit 1016. Another type of event to be detected by the event detector 1023 is an event which serves as a trigger for causing the information manager 1025 to specify a measurement position at which the above-described body sound information has been collected. For example, the information manager 1025 is able to specify a measurement position by extracting information for specifying a measurement position contained in the above-described specific image, or by determining how many times the above-described specific instruction signal, voice signal, or information has been input so far from the start of auscultation. In this case, auxiliary information necessary to specify a measurement position will be referred to as “auxiliary information”.

The body-sound-information obtaining section 1024 controls the wireless communication unit 1016 so as to obtain body sound information of a patient received by the wireless communication unit 1016. If necessary, the body-sound-information obtaining section 1024 may perform appropriate information processing, such as converting a data format of body sound information into a format that can be handled by the information manager 1025, or converting header information appended to body sound information into a format that can be recognized by the information manager 1025.

The information manager 1025 is used for managing obtained body sound information, in particular, it is used for linking body sound information to measurement position information. More specifically, concerning body sound information obtained by the body-sound-information obtaining section 1024, the information manager 1025 specifies a position on the body surface of a patient at which the body sound information, which is sound information, has been collected. Then, the information manager 1025 links measurement position information indicating this measurement position to the body sound information and stores the body sound information in the body-sound-information storage section 1031. The body-sound-information storage section 1031 may be a storage device that stores body sound information temporarily in a non-volatile manner. In this case, body sound information stored in the body-sound-information storage section 1031 is transferred, at a suitable timing, to an external storage device or the management server 1004 shown in FIG. 8 by an information transfer controller (information transfer control means), which is not shown.

As long as auscultation assisting information is being output by the output control section 1022, the information manager 1025 can assume that auscultation is being conducted in accordance with this auscultation assisting information. More specifically, since auscultation assisting information includes measurement position information, the information manager 1025 is able to identify that the body sound information has been collected at a position indicated by one of the items of measurement position information included in the auscultation assisting information which is being output. Additionally, as discussed above, the information manager 1025 is able to specify a measurement position by receiving information indicating the occurrence of a specific event detected by the event detector 1023.

That is, on the basis of the content of auscultation assisting information which is being selected and output and information obtained, as a trigger, upon the occurrence of a specific event detected while the auscultation assisting information is being output, the information manager 1025 is able to specify measurement position information indicating a position at which body sound information has been collected by a digital stethoscope and to link the body sound information to the measurement position information.

With the above-described configuration, in the information management apparatus 1100 implemented by a widely used device, such as a PC or a smartphone, the auscultation-assisting-information selector 1021 selects an item of auscultation assisting information suitable for patient attributes. The output control section 1022 then displays the selected item of auscultation assisting information so that the operator U can visually check it. Meanwhile, the information manager 1025 specifies a measurement position of the obtained body sound information, on the basis of auscultation assisting information which is being output and information obtained, as a trigger, upon the occurrence of a specific event detected while the auscultation assisting information is being output. The information manager 1025 is able to link the measurement position information to the obtained body sound information and to store the body sound information in the body-sound-information storage section 1031.

As a result, by using the information management apparatus of the present invention, it is possible to allow an operator to perform measurement by using a digital stethoscope without using a special device involving a complicated operation and to easily link collected body sound information to measurement position information concerning the body sound information.

### [Information Management Function Using Projector]

In this embodiment, the information management apparatus 1100 is implemented by a device (such as a smartphone) including the projecting unit 1014 (projector function) and the imaging unit 1017. If the information management function of the present invention is implemented by using the information management apparatus 1100, more advantages can be achieved in addition to the above-described advantages. Hereinafter, a description will be given, by using a specific example, of a case in which the auscultation system 1200 is implemented by using the imaging unit 1017 and the projecting unit 1014 (projector function) of the information management apparatus 1100.

In this embodiment, the patient information obtaining section 1020 obtains patient information as live view images captured by the imaging unit 1017. For example, if the operator U operates the information management apparatus 1100 and selects an auscultation application, the imaging unit 1017 is started and begins to capture images of a patient. It is sufficient that the operator U merely sets the information management apparatus 1100 at a suitable position so that the patient P can be contained within a capturing range of the imaging unit 1017. With this configuration, the operator U does not have to manually input patient information, thereby further reducing complicated operations.

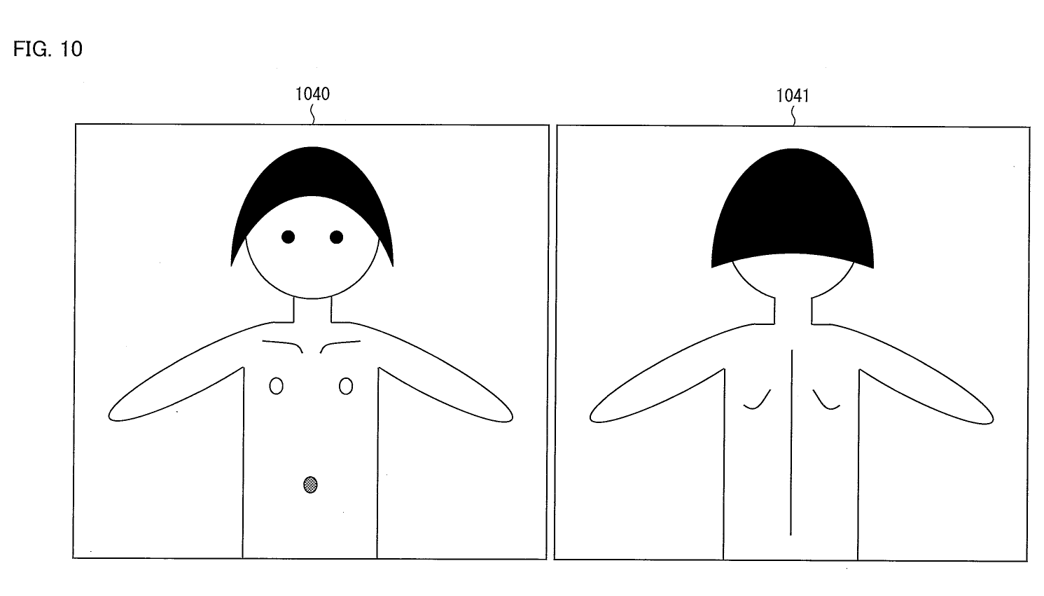

FIG. 10 illustrates examples of live view images of the patient P captured by the imaging unit 1017. A patient image 1040 is an image obtained by imaging a front side of the patient P, while a patient image 1041 is an image obtained by imaging a back side of the patient P. If the operator U wishes to examine the front side of the patient P, the operator U images the front side of the patient P by using the information management apparatus 1100.

In this embodiment, the patient information obtaining section 1020 obtains live view images sequentially supplied from the imaging unit 1017 and supplies the live view images to the auscultation-assisting-information selector 1021. These live view images may be displayed in the display unit 1012 of the information management apparatus 1100.

In this embodiment, the auscultation-assisting-information selector 1021 controls an image recognition processor (image recognition processing means), which is not shown, so as to implement an image recognition function. Then, the auscultation-assisting-information selector 1021 is able to identify the body-build of a patient as patient attributes. More specifically, the auscultation-assisting-information selector 1021 performs image recognition processing on a live view image obtained by the patient information obtaining section 1020, and extracts characteristic points detected from the live view image. Characteristic points to be detected are defined in advance by the image recognition function. In this embodiment, for identifying the overall shape of the body of a patient, lines of a neck, shoulders, arms, side, and waist may be extracted as characteristic points. These lines may be extracted by a known edge detection technique.

It is preferable that images of the patient P is taken in accordance with predetermined rules. Predetermined rules are, for example, that a patient always stands in front of a black board (may be any color as long as a skin color stands out against the color in a background) and stretches his/her arms (grips handles), such as in a situation where the patient P is subjected to chest X-ray photography. If image recognition is performed on a patient image captured in accordance with such rules, the auscultation-assisting-information selector 1021 is able to more precisely extract the lines of the neck, shoulders, arms, side, and waist of the patient as the characteristic points without misrecognizing them.

It may be possible that, before taking an image of a patient, the operator U input an instruction, into the information management apparatus 1100, whether to examine the front side or the back side of the patient. In this case, if the front side of the patient is specified, the auscultation-assisting-information selector 1021 may extract, as the characteristic points, characteristic parts of the front side of the upper body of a human, such as the clavicle, ribs, nipples, and navel. If the back side of the patient is specified, the auscultation-assisting-information selector 1021 may extract, as the characteristic points, characteristic parts of the back side of the upper body of a human, such as scapulae and dorsal muscles. With this arrangement, characteristic points to be detected are restricted in advance, thereby reducing the load in image recognition processing.

Alternatively, the auscultation-assisting-information selector 1021 may check for all the characteristic points defined in advance from a live view image and determine, on the basis of checking results, whether the patient image indicates the front side or the back side of the patient. For example, if nipples and a navel are detected from the patient image 1040 shown in FIG. 10, the auscultation-assisting-information selector 1021 may determine that the patient image 1040 is an image of the front side.

Alternatively, the auscultation-assisting-information selector 1021 may determine whether the patient image indicates the front side or the back side of a patient according to whether the face of the patient has been recognized or a substantially solid color, such as a hair color or a skin color of the patient, has been recognized. In this manner, since the auscultation-assisting-information selector 1021 determines from a patient image whether the image indicates the front side or the back side of a patient, the operator U does not have to manually specify whether the front side or the back side of a patient will be subjected to auscultation. As a result, complicated operations can further be reduced.

Then, the auscultation-assisting-information selector 1021 estimates the body-build of a patient on the basis of positional relationships among extracted characteristic points (distances among the characteristic points) and the distance between the imaging unit 1017 and a subject (in this example, the patient P).

For example, from the patient image 1040 indicating the front side of a patient, the size of a triangle using three points, such as the nipples and the navel, as vertexes and the distance between the imaging unit 1017 and the subject are determined, and then, on the basis of the size and the distance, the auscultation-assisting-information selector 1021 is able to estimate the size and the proportion ratio of the waist and the height of the patient P.

By performing the above-described image recognition processing, the auscultation-assisting-information selector 1021 estimates patient attributes. For example, as the attributes of the patient P indicated in the patient image 1040, the auscultation-assisting-information selector 1021 determines three items, such as (1) front side, (2) height: 150 to 170 cm, and (3) body type: normal weight.

In this embodiment, the auscultation-assisting-information selector 1021 performs image recognition on a live view image captured by the patient-image obtaining section 1020. Alternatively, the auscultation-assisting-information selector 1021 may process a patient image, which is a still image, captured by the imaging unit 1017. In this case, however, a patient may feel uncomfortable about storing an image captured by the imaging unit 1017 as data. Such personal information should also be treated as discreet as possible. Accordingly, by considering patient's feelings, it is necessary that only live view images be obtained so that data of patient images will not be permanently stored in the information management apparatus 1100, or that the information management apparatus 1100 be constructed such that data of patient images which are not necessary any more are deleted immediately after it has been used.

Then, on the basis of the determined patient attributes, the auscultation-assisting-information selector 1021 selects auscultation assisting information suitable for the patient attributes. In this embodiment, in the auscultation-assisting-information storage section 1030, several items of auscultation assisting information are stored in accordance with the orientation (front side or back side) of a patient and the body-build of a patient. The auscultation-assisting-information selector 1021 selects and reads an item of auscultation assisting information associated with the orientation and the body-build of the patient determined as described above from the auscultation-assisting-information storage section 1030.

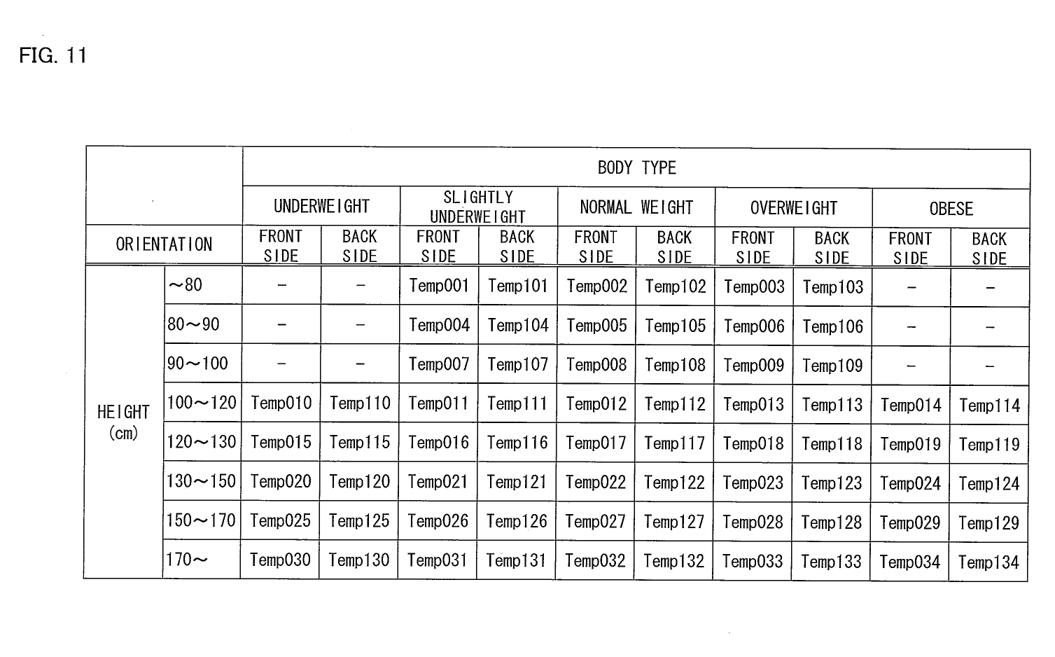

FIG. 11 is a table illustrating a specific example of a data structure of an auscultation-assisting-information database stored in the auscultation-assisting-information storage section 1030.

As shown in FIG. 11, in the auscultation-assisting-information database, plural items of auscultation assisting information are stored in association with the patient attributes, that is, the orientation, height, and body type.

The auscultation-assisting-information database shown in FIG. 11 is indicated by a data structure in a table format by way of example, and it is not intended to restrict the data structure of the auscultation-assisting-information database. The auscultation-assisting-information database may be formed in any data structure as long as the association between patient attributes and an item of auscultation assisting information to be selected is recognizable by the auscultation-assisting-information selector 1021. The following embodiments will also be treated in a similar manner.

More specifically, if the auscultation-assisting-information selector 1021 has determined, as patient attributes, “front side”, “150 to 170 cm”, and “normal weight” from the patient image 1040, it selects auscultation assisting information “Temp027” that matches these attributes. The selected auscultation assisting information “Temp027” includes optimal and useful information for conducting auscultation on the patient P indicated in the patient image 1040. In the example shown in FIG. 11, in the patient attribute “height”, “− **” means less than ** cm, and “XX −” means XX cm or greater.

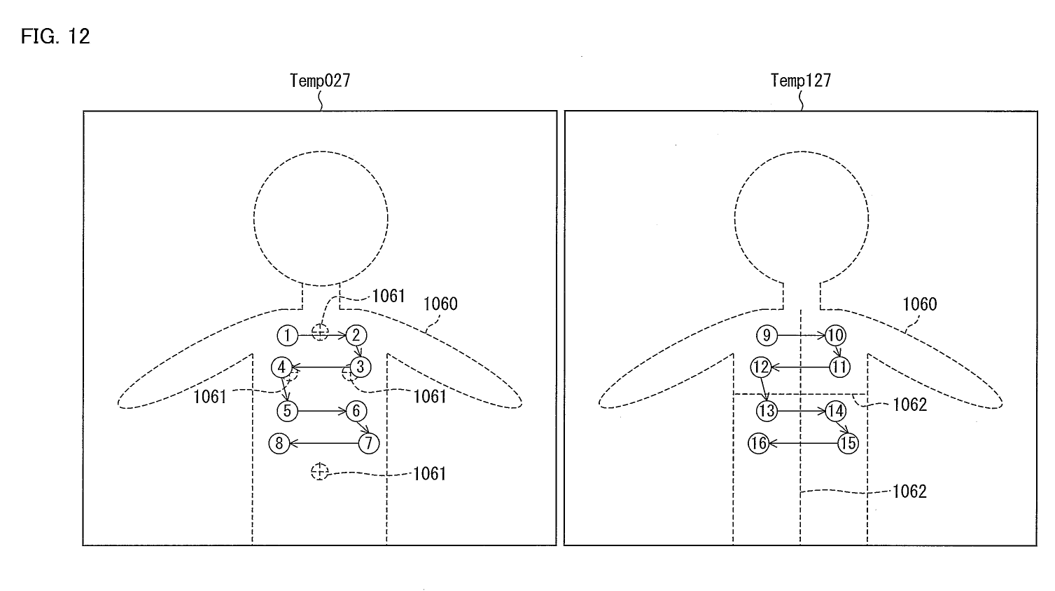

FIG. 12 illustrates specific examples of auscultation assisting information stored in the auscultation-assisting-information storage section 1030. The auscultation assisting information represented by Temp027 shown in FIG. 12 is auscultation assisting information selected as a result of the auscultation-assisting-information selector 1021 performing image recognition processing on the patient image 1040. The auscultation assisting information represented by Temp127 shown in FIG. 12 is auscultation assisting information selected as a result of the auscultation-assisting-information selector 1021 performing image recognition processing on the patient image 1041.

In this embodiment, auscultation assisting information is image data, and measurement position information is defined by using a contour 1060 and characteristic points of the body of a patient as reference positions. The characteristic points may be represented on the basis of reference points 1061 or reference lines 1062.

In this embodiment, measurement position information is represented by symbols disposed at least on the basis of the contour 1060 of a patient, and the reference points 1061 and/or the reference lines 1062. In the examples shown in FIG. 12, the circles indicate measurement position information.

The auscultation assisting information may also include measurement order information which specifies the order of auscultation. In the examples shown in FIG. 12, measurement order information is represented by the arrows linking the circles or the numbers appended to the circles. The measurement order information represented by the numbers in the circles can also be utilized as measurement position identification information for individually identifying measurement positions.

By visually checking this auscultation assisting information, the operator U can understand in which order and at which positions of the patient P the digital stethoscope 1003 should be applied.

In this embodiment, a description has been given, assuming that auscultation assisting information is an image. However, the data format of auscultation assisting information in the present invention is not particularly restricted. Auscultation assisting information may simply be position information (coordinate information) in predetermined coordinate systems. For example, the middle point between nipples may be set to be an origin (0, 0), and measurement position information may be represented by an X coordinate value and a Y coordinate value.

In this embodiment, the output control section 1022 functions as a projection controller that outputs a video signal of a selected item of auscultation assisting information to the projecting unit 1014 and that causes the projecting unit 1014 to enlarge and project the video signal on an external light receiver.

The output control section 1022 may directly output an image of auscultation assisting information, such as that shown in FIG. 12, as a projection image. Alternatively, the output control section 1022 may generate a projection image by superposing measurement position information (and measurement order information) contained in auscultation assisting information on a patient image obtained by the patient information obtaining section 1020.

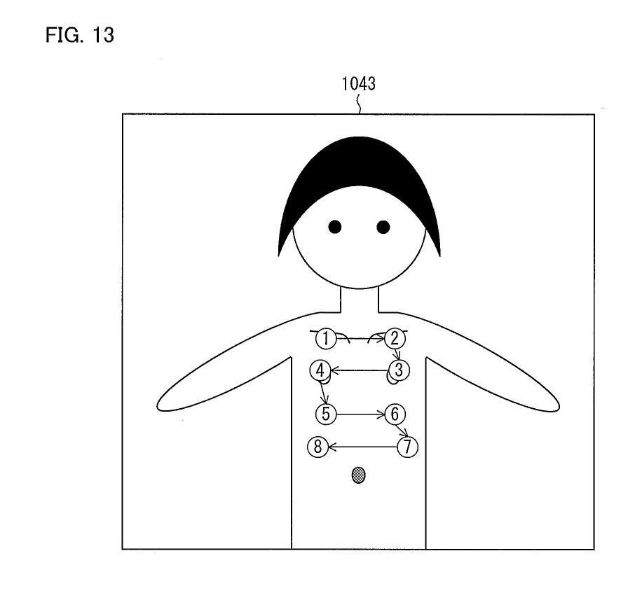

FIG. 13 illustrates a specific example of a projection image generated by the output control section 1022.

It is now assumed that the auscultation-assisting-information selector 1021 has selected auscultation assisting information represented by Temp027 shown in FIG. 12 on the basis of the patient image 1040 shown in FIG. 10.

The output control section 1022 superposes measurement position information (circles) included in the auscultation assisting information represented by Temp027 on the patient image 1040. The output control section 1022 may also superpose measurement order information (numbers appended to the circles and the arrows) included in the auscultation assisting information on the patient image 1040. By determining superpose positions in a manner such that the contour 1066, and the reference points 1061 or the reference lines 1062 of the auscultation assisting information can be adjusted to corresponding portions of the patient image contained in the patient image 1040 as precise as possible, the output control section 1022 can correctly superpose the measurement position information (circles) on the body surface of the patient P in the patient image 1040. In this manner, the output control section 1022 generates a projection image 1043, as shown in FIG. 13. The projection image 1043 is generated by superposing the measurement position information and the measurement order information of the auscultation assisting information represented by Temp027 on the patient image 1040.

A video signal indicating a projection image (auscultation assisting information itself or a generated image, such as the projection image 1043) is output from the output control section 1022 to the projecting unit 1014. The projecting unit 1014 enlarges and projects the received video signal on a light receiver (such as a wall or a screen).

By visually checking the projection image, the operator U can understand at which positions the digital stethoscope 1003 should be applied in accordance with the body-build of a patient P. If measurement order information is included in the projection image, the operator U can understand in which order the digital stethoscope 1003 should be applied.

In this manner, if the projection image 1043 obtained by superposing measurement position information on a video image indicating an actual patient using the patient image 1040 is output, the operator U can more precisely identify measurement positions on the body surface of the patient P, compared with a case in which Temp027 indicated on a model is output.

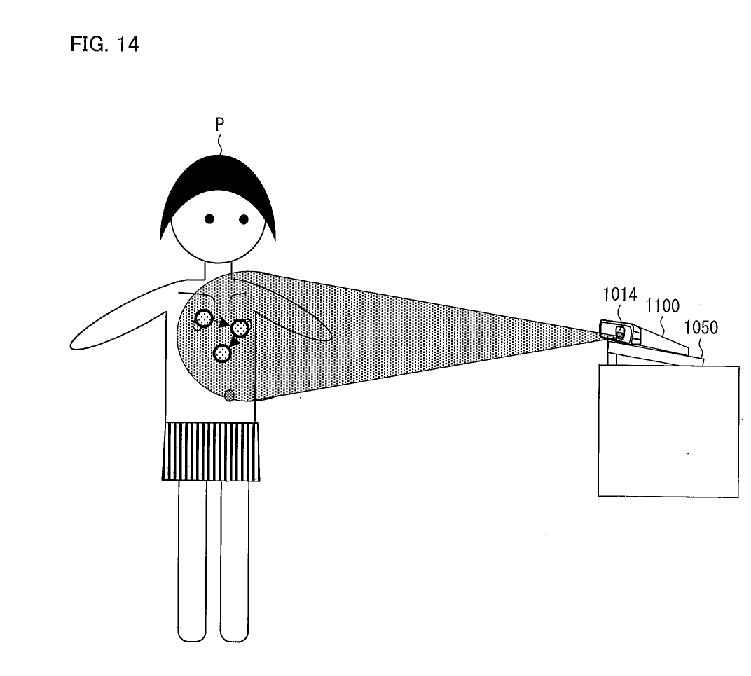

It is preferable that a projection image output from the projecting unit 1014 is directly projected on the actual body of the patient P. FIGS. 14 through 16 show modes in which a projection image is directly projected on the actual body of the patient P.

The operator U adjusts the distance between the patient P and the information management apparatus 100 and the orientation thereof so that an optical video image output from the projecting unit 1014 can be projected on the patient P. As shown in FIG. 14, an installation table 1050 may be set, and the information management apparatus 1100, which serves as a projector, may be fixed on the installation table 1050.

Alternatively, the operator U may adjust the scaling factor of a projection image by using the operation unit 1013 (such as a dial) or the input unit 1011 of the information management apparatus 1100.

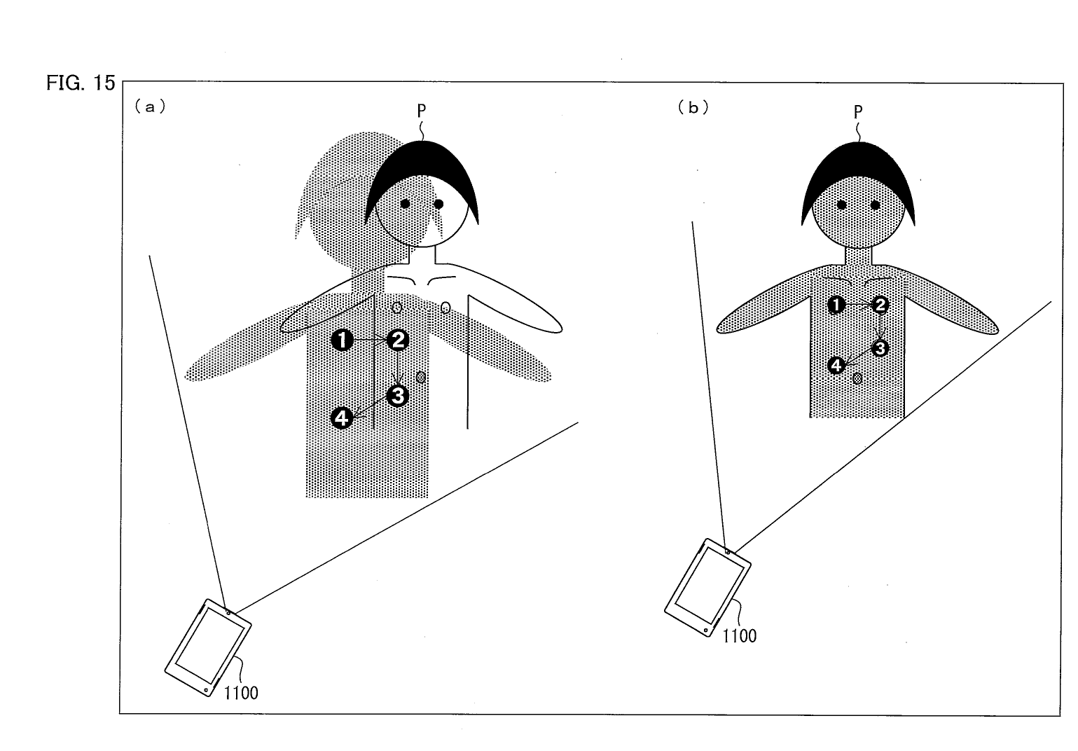

For example, there may be a case in which, as shown in part (a) of FIG. 15, the size of a projection image does not match the size of the patient P since a sufficient distance between the information management apparatus 1100 and the patient P is not secured. In this case, the operator U may reduce the scaling factor of the projection image by operating the operation unit 1013 to such a degree that the contour of the projection image 1043 matches the contour of the actual body of the patient P. The optimal projection state is, as shown in part (b) of FIG. 15, that the contour of the projection image 1043 exactly matches the contour of the actual body of the patient P.

With the above-described configuration, the operator U can intuitively understand measurement positions by the measurement position information projected on the body surface of the patient P. By comparison with a mode in which measurement position information and measurement order information are displayed in a place other than the body of a patient, by using a mode in which measurement position information and measurement order information are displayed on the body surface of a patient, the operator U can be prevented from misrecognizing the measurement positions and the measurement order. As a result, the frequency with which auscultation failures occur due to incorrect operations can be significantly reduced.

Additionally, projected auscultation assisting information is an item of information suitable for the body-build of a patient. Thus, projected measurement positions highly precisely reproduce portions of the patient to be subjected to auscultation.

In this manner, since the information management apparatus 1100 of the present invention displays measurement position information suitable for the body-build of a patient on the body surface of the patient, the operator U can intuitively understand measurement positions.

Accordingly, even if the operator U does not have medical expertise, the operator U is able to perform correct auscultation.

If the operator U performs auscultation by applying the digital stethoscope 1003 while being in the way between the patient P and the information management apparatus 1100, measurement position information is not projected on the patient P since a shadow of the operator U is cast on the patient P.

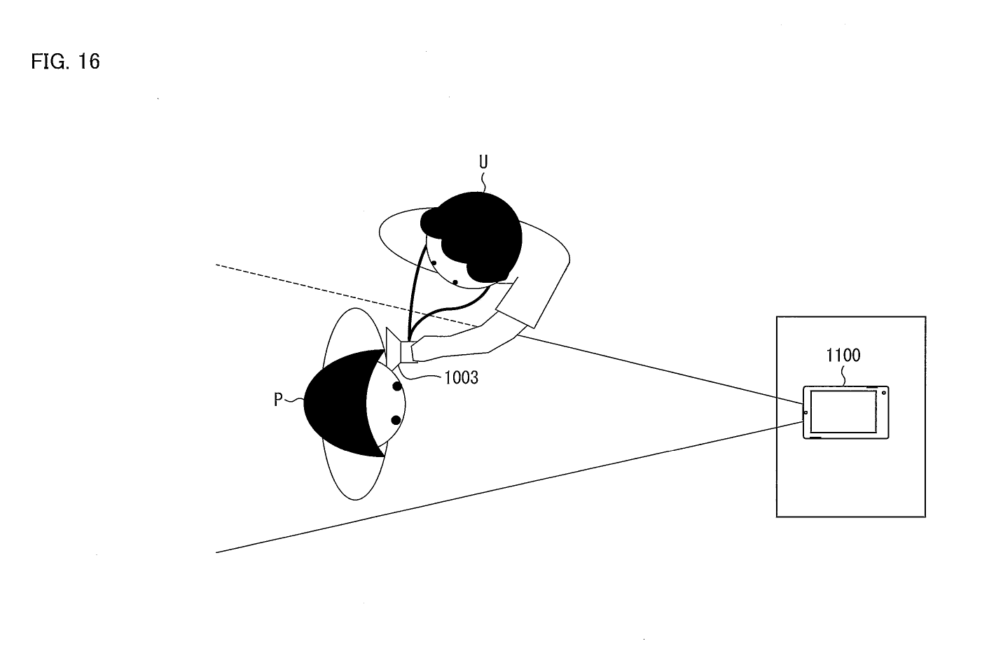

In order to avoid such a problem, instead of conducting auscultation at a position in front of the patient P, as shown in FIG. 16, the operator U has to conduct auscultation by stretching a hand from a position at a side of the patient P and applying the digital stethoscope 1003 to measurement positions. There may be many cases in which a patient subjected to auscultation suffers from a disease which causes droplet infection by coughing, such as a respiratory system disease. Accordingly, it is recommended that, when examining the front side of a patient, an operator conduct auscultation from a side of the patient so as not to be exposed to coughs emitted from the patient. An auscultation mode of the present invention is not inconsistent with such a recommended auscultation mode.

In this embodiment, by identifying measurement position information and measurement order information of auscultation assisting information which is being output from the output control section 1022, the information manager 1025 is able to specify a measurement position at which body sound information has been collected by using the digital stethoscope 1003 and to link the measurement position information to the body sound information and manage the body sound information. More specifically, linking of measurement position information to body sound information will be performed in the following procedure.

In this embodiment, by way of example, the digital stethoscope 1003 collects a continuous sound waveform for one measurement position, and transfers body sound information concerning one measurement position as one file to the information management apparatus 1100. In this embodiment, the digital stethoscope 1003 transfers body sound information in real time online to the information management apparatus 1100 during a period from the start to the end of the collection of sound waveforms.

The event detector 1023 detects, via the wireless communication unit 1016, that the reception of body sound information is started as a specific event.

As the occurrence of the above-described event as a trigger, the information manager 1025 obtains information (auxiliary information) necessary to specify a measurement position in the started auscultation. More specifically, the information manager 1025 determines how many files concerning the body sound information have been received so far since the output control section 1022 output a projection image (for example, the projection image 1043 shown in FIG. 13), and obtains such information as auxiliary information.

For example, if the number of files concerning the body sound information received and stored since the output control section 1022 output the projection image 1043 is zero, the information manager 1025 determines that the reception of a first file (a first item of body sound information) is started.

Then, the information manager 1025 refers to auscultation assisting information output from the output control section 1022. The information manager 1025 then specifies that the first item of body sound information has been collected at a measurement position indicating the number 1 in the measurement order information (the circle indicating the number 1 in the example shown in FIG. 12).

Upon completion of the reception of the first item of body sound information, the information manager 1025 links the measurement position information “1” specified as described above to the body sound information and stores the body sound information and the measurement position information in the body-sound-information storage section 1031.

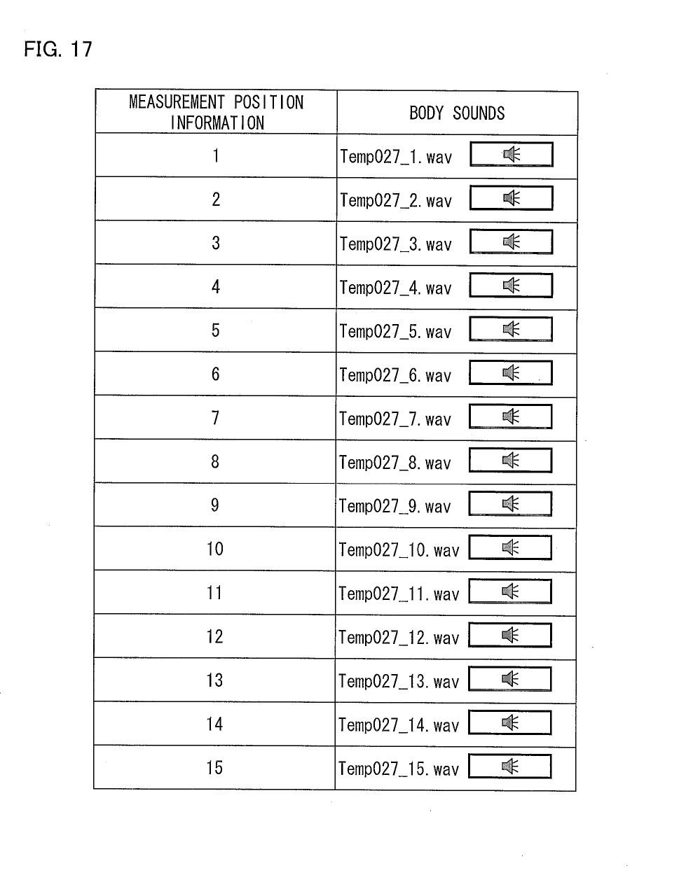

FIG. 17 illustrates a specific example of measurement position information and body sound information stored in the body-sound-information storage section 1031. In FIG. 17, plural items of body sound information are indicated by a data structure in a table format only by way of example. Body sound information may be stored in any data structure as long as linking relationships between measurement position information and body sound information are recognizable by the information management apparatus 1100. The following embodiments will also be treated in a similar manner.

As shown in FIG. 17, the information manager 1025 stores body sound information as one file for one measurement position in the body-sound-information storage section 1031. In this case, the information manager 1025 stores body sound information by linking measurement position information specified as described above to the body sound information. Although the file name appended to body sound information is not particularly restricted, it is preferable that a file name indicating which auscultation assisting information has been used to conduct auscultation is appended. With this arrangement, it is possible to recognize that, for example, body sound information having a file name “Temp027_1.wav” linked to measurement position information “1”, is body sound information obtained at a position of the circle with the number 1 in the auscultation assisting information represented by Temp027.

In the example shown in FIG. 17, only measurement position information is linked to body sound information. However, information linked to body sound information is not restricted to measurement position information. The information manager 1025 may also link another item of information, such as patient information or a measurement date, to body sound information.

Body sound information stored in the body-sound-information storage section 1031 by the information manager 1025 is transferred to, for example, the management server 1004, by an information transfer controller (not shown). The management server 1004 is able to receive and store body sound information, together with measurement position information. Accordingly, by accessing the management server 1004, a physician D who is not in the clinic 1001 is able to play back body sound information after recognizing at which measurement position the body sound information has been collected.

### [Processing Flow]

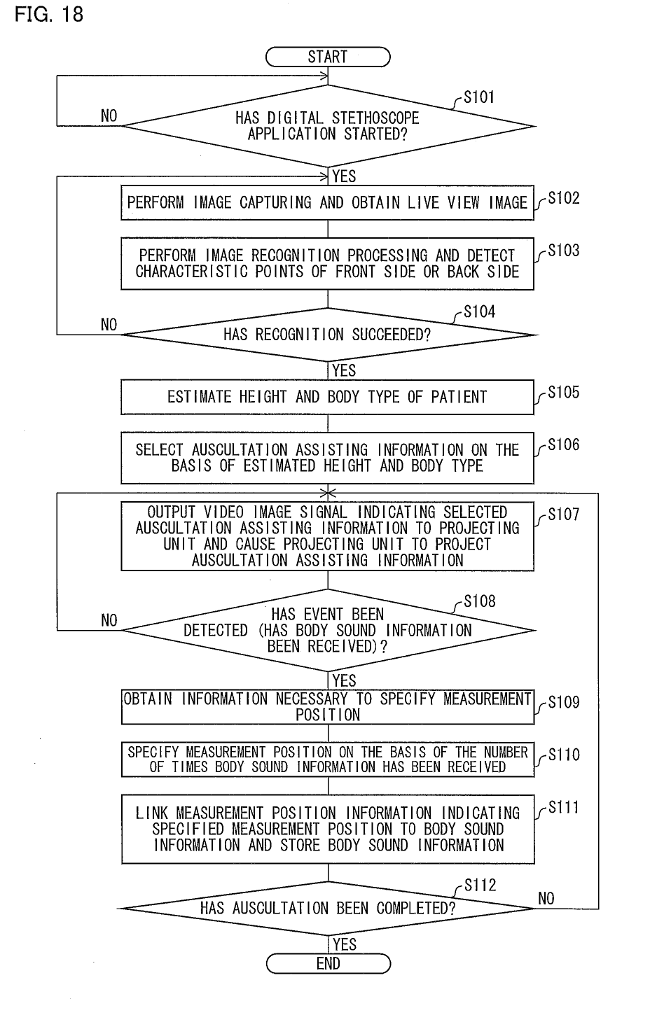

FIG. 18 is a flowchart illustrating a flow of information management processing performed by the information management apparatus 1100.

In the clinic 1001, the operator U operates the information management apparatus 1100 (for example, by touching an icon) to start an application concerning a digital stethoscope. If the digital stethoscope application is started (YES in S101), the imaging unit 1017 performs image capturing to obtain a live view image. In this case, the operator U adjusts the orientation of the information management apparatus 1100 so that the upper body of the patient P will be contained in a capturing range of the imaging unit 1017. The patient-image obtaining section 1020 obtains a live view image (patient image indicating a patient) generated by performing image capturing by the imaging unit 1017 (S102).

The auscultation-assisting-information selector 1021 performs image recognition processing on the obtained patient image so as to detect characteristic points necessary to determine patient attributes (S103). In this case, the auscultation-assisting-information selector 1021 may determine, on the basis of the detected characteristic points, a patient attribute, that is, whether the patient image indicates a front side or a back side of the patient.

If necessary characteristic points have been detected and the orientation of the patient (front side or back side) has been determined, it means that image recognition has succeeded. If image recognition has not succeeded (NO in S104), the imaging unit 1017 continues image capturing until necessary information can be obtained from a live view image (S102).

If image recognition has succeeded (YES in S104), the auscultation-assisting-information selector 1021 estimates the remaining patient attributes (S105). More specifically, the auscultation-assisting-information selector 1021 estimates the height and the body type of the patient and so on, on the basis of the detected characteristic points. The auscultation-assisting-information selector 1021 then selects an item of auscultation assisting information suitable for the patient from among items of auscultation assisting information stored in the auscultation-assisting-information storage section 1030, on the basis of the estimated orientation, height, and weight of the patient (S106).

The output control section 1022 generates a video image signal indicating a projection image, on the basis of the item of auscultation assisting information selected by the auscultation-assisting-information selector 1021, and outputs the generated video image signal to the projecting unit 1014 (S107). Light of the video image signal output from the projecting unit 1014 is projected on the body surface of the patient P. In this case, as shown in part (b) of FIG. 15, the operator U adjusts the installation position and the orientation of the information management apparatus 1100 and the scaling factor of the projecting unit 1014 so that the size of the contour of a human model of the projection image will match the actual size of the patient P.

The operator U conducts auscultation by using the digital stethoscope 1003 in accordance with measurement position information and measurement order information projected on the body of the patient P.

Upon detection of a specific event by the event detector 1203 (YES in S108), the information manager 1025 obtains auxiliary information necessary to specify a measurement position for the currently collected body sound information (S109). More specifically, the event detector 1023 detects, as a specific event, that body sound information has been received from the digital stethoscope 1003 (S108). Then, the information manager 1025 determines how many times body sound information has been received so far since the projection image was output in S107, and specifies the determined number of times as auxiliary information (S109).

Then, the information manager 1025 specifies a measurement position of the currently collected item of body sound information, on the basis of the number of times body sound information has been received and the currently output auscultation assisting information (S110). For example, if the number of times body sound information has been received is zero, the information manager 1025 can specify the measurement position of this first item of body sound information to be a first measurement position in the auscultation assisting information.

Then, the information manager 1025 links measurement position information concerning the specified measurement position to the received item of body sound information and stores them in the body-sound-information storage section 1031 (S111).

Auscultation is repeated the same number of times as that of the items of measurement position information included in the auscultation assisting information. Accordingly, if auscultation has not been completed for all the measurement positions (NO in S112), the projecting unit 1014 continues a projecting operation (S107), and the event detector 1023 waits until a subsequent item of body sound information has been received.

If body sound information has been collected for all the measurement positions and auscultation has been completed (YES in S112), the information management apparatus 1100 may terminate the entire information management processing. Alternatively, in the information management apparatus 1100, an information transfer controller (not shown) may transfer body sound information stored by the information manager 1025 to the management server 1004 in the support center 1002 in the state in which the measurement position information is linked to the body sound information.

With the above-described configuration and method, in the information management apparatus 1100 implemented by a widely used device, such as a PC or a smartphone, the auscultation-assisting-information selector 1021 selects an item of auscultation assisting information suitable for patient attributes (the body-build of a patient). The output control section 1022 then displays the selected item of auscultation assisting information so that the operator U can visually check it. More specifically, the output control section 1022 projects the auscultation assisting information on the body surface of the patient P such that the size of the auscultation assisting information matches the size of the body of the patient P.

Meanwhile, the information manager 1025 specifies a measurement position of the obtained body sound information, on the basis of the auscultation assisting information which is being projected and information obtained, as a trigger, upon the occurrence of a specific event detected while the auscultation assisting information is being projected. The information manager 1025 links the measurement position information to the obtained body sound information and stores them in the body-sound-information storage section 1031.

The operator U does not have to perform a complicated input operation for selecting an optimal item of auscultation assisting information, and instead, the operator U only has to make adjustment so that a patient will be contained in a capturing range of the imaging unit 1017. Additionally, since the same size of auscultation assisting information as that of the body of the patient P is displayed on the body surface of the patient P, the operator U is able to highly precisely perform auscultation without misrecognizing measurement positions and measurement order. Then, the information manager 1025 of the information management apparatus 1100 specifies a measurement position of body sound information, on the basis of measurement position information included in the auscultation assisting information which is being output and the number of times the body sound information has been received. Finally, the information manager 1025 links measurement position information indicating the specified measurement position to the body sound information and stores them in a storage unit.

As a result, by using the information management apparatus of the present invention, it is possible to allow an operator to perform measurement by using a digital stethoscope without using a special device involving a complicated operation and to easily link collected body sound information to measurement position information concerning the body sound information.

### First Modified Example

The output control section 1022 of this embodiment may instruct the operator U to change a capturing range of the imaging unit 1017 so as to obtain a live view image from which the auscultation-assisting-information selector 1021 can estimate the body-build of a patient.

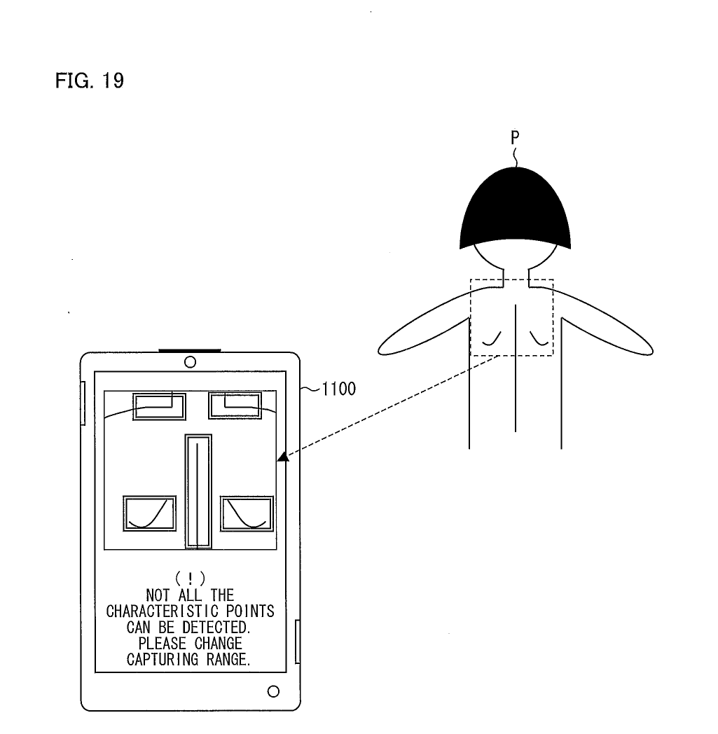

FIG. 19 illustrates an example in which a screen for instructing an operator to change a capturing range is displayed in the display unit 1012.

As shown in FIG. 19, the output control section 1022 displays a live view image currently obtained by the patient information obtaining section 1020 in the display unit 1012. Then, a case in which necessary characteristic points have not been detected from the live view image, that is, a case in which the contour of the patient P has not been captured, as shown in FIG. 19, is assumed. In this case, the output control section 1022 determines that image recognition has not succeeded, and displays, as shown in FIG. 19, a message instructing the operator U to change the capturing range in the display unit 1012.

This makes it possible for the operator U to speedily respond to this message and handle such a situation to obtain a suitable live view image.

### Second Modified Example

When examining a patient in the clinic 1001, it is necessary, in most cases, to conduct auscultation on both of the front side and the back side of the patient. In this embodiment, the auscultation-assisting-information selector 1021 selects auscultation assisting information for each of the front side and the back side of a patient. For selecting auscultation assisting information, the auscultation-assisting-information selector 1021 performs relatively high-load image recognition processing. It is preferable that the load of such image recognition processing is reduced.

In this embodiment, therefore, it is possible to determine a rule that, when conducting auscultation operations on one patient, image capturing is always started from the front side of the patient. Then, when a patient image for this patient is input for the first time, the auscultation-assisting-information selector 1021 may immediately start detecting characteristic points of the front side of a body, assuming that the input patient image is an image of the front side. Then, when a patient image for this patient is input for the second time, the auscultation-assisting-information selector 1021 can immediately start detecting characteristic points of the back side, assuming that the input patient image is an image of the back side of the patient.

With this configuration, it is possible to omit high-load processing for checking for all possible characteristic points first and then determining whether a patient image indicates the front side or the back side of the patient. Additionally, a patient image of the front side of a patient has more distinct characteristic points (such as nipples and a navel) than that of the back side of the patient. Thus, the body-build of a patient can be estimated faster with a lower load.

On the basis of a patient image which is assumed as an image of the front side of a patient, the auscultation-assisting-information selector 1021 immediately estimates the body-build of the patient and selects front-side auscultation assisting information suitable for the estimated body-build of the patient. At this time, together with the front-side auscultation assisting information, the auscultation-assisting-information selector 1021 may also select back-side auscultation assisting information suitable for the body-build of the patient estimated from the patient image of the front side.

The back side and the front side are two sides of the same patient, and thus, the two sides do not differ considerably since the size of the patient is the same. Accordingly, the auscultation-assisting-information selector 1021 may select back-side auscultation assisting information on the basis of the body-build of the patient estimated from a patient image of the front side of the patient. With this arrangement, even if image recognition processing for a patient image of the back side of a patient is entirely omitted, correct items of auscultation assisting information suitably used for the front side and the back side of a patient can be selected.

### Third Modified Example

In the above-described description, upon the occurrence of an event indicating that body sound information has been obtained, the information manager 1025 specifies a measurement position of the body sound information.

However, the information manager 1025 is not restricted to this configuration, and may specify, in advance, a measurement position at which body sound information will be collected subsequently and may present the specified measurement position to the operator U.

When a specific item of auscultation assisting information is output from the output control section 1022, the information manager 1025 determines that measurement should be started from a first measurement position indicated in measurement order information. Then, the information manager 1025 instructs the output control section 1022 to highlight a first item of measurement position information indicated in the measurement order information. The output control section 1022 outputs auscultation assisting information and also highlights an item of measurement position information only specified by the information manager 1025. For example, the output control section 1022 may display the specified item of measurement position information with a cursor, or in a larger size or in a different color. Alternatively, the output control section 1022 may cause the specified item of measurement position information to blink. The operator U can then recognize that the highlighted measurement position is a position at which the operator U is supposed to apply a stethoscope subsequently.

Upon detecting an event, by the event detector 1023, that the reception of a first item of body sound information has finished, the information manager 1025 determines that auscultation at the first measurement position has finished and a second item of measurement position information will be highlighted. At this time, the information manager 1025 instructs the output control section 1022 to change the item of measurement position information to be highlighted from the first item of measurement position information to the second item of measurement position information.

### Fourth Modified Example

In this embodiment, the information manager 1025 determines the start and the end of an auscultation operation at each measurement position, on the basis of an event detected by the event detector 1023 that the reception of body sound information has started and an event detected by the event detector 1023 that the reception of body sound information has finished.

However, the approach to determining the start or the end of an auscultation operation by the information manager 1025 is not restricted to the above-described approach.

For example, when starting or finishing an auscultation operation at one position, it may be possible that the operator U flick the touch panel of the information management apparatus 1100. In this case, the event detector 1023 detects, as an event, that the touch panel has been flicked via the input unit 1011. This makes it possible for the information manager 1025 to determine the start or the end of an auscultation operation at one position.

Alternatively, it may be possible that a button for inputting the start or the end of an auscultation operation be provided in the digital stethoscope 1003 and the operator U press the button of the digital stethoscope 1003 when starting or finishing an auscultation operation at one position. The event detector 1023 then detects, as an event, via the wireless communication unit 1016 that the button of the digital stethoscope 1003 has been pressed. This makes it possible for the information manager 1025 to determine the start or the end of an auscultation operation at one position.

Alternatively, it may be possible that a contact sensor be provided on a face of the digital stethoscope 1003 which is brought into contact with the body surface of a patient and that the sensor detect whether or not the digital stethoscope 1003 is in contact with the body surface of a patient. Then, the event detector 1023 detects, via the wireless communication unit 1016, a time point at which the digital stethoscope 103 starts to contact the body surface and a time point at which the digital stethoscope 1003 starts to be released from the body surface. This makes it possible for the information manager 1025 to determine the start or the end of an auscultation operation at one position. The sensor of the digital stethoscope 1003 may detect whether the digital stethoscope 1003 stays still on the body surface of a patient or is moving to a subsequent measurement position on the body surface. The event detector 1023 detects, via the wireless communication unit 1016, a time point at which the digital stethoscope 1003 starts to stay still, starts to move, starts to stay still again, and so on. This makes it possible for the information manager 1025 to determine that a time point at which the digital stethoscope 1003 starts to stay still and a time point at which the digital stethoscope 1003 finishes staying still correspond to the start and the end of an auscultation operation at one position.

The imaging unit 1017 may image the front side or the back side of a patient P on which the projection image 1043 output from the projecting unit 1014 is projected. In this case, a live view image obtained by the imaging unit 1017 is subjected to image recognition by an image recognition processor (not shown). The event detector 1023 detects, as an event, on the basis of the results of image recognition processing, that the digital stethoscope 1003 has been staying still for a certain period or longer or that the digital stethoscope 1003 has finished staying still and started to move to another position. This makes it possible for the information manager 1025 to determine that a certain period or longer for which the digital stethoscope 1003 stays still corresponds to the start and the end of an auscultation operation at one position.

### Fifth Modified Example

In this embodiment, auscultation assisting information stored in the auscultation-assisting-information storage section 1030 may include measurement position identification information instead of measurement order information. Measurement position identification information indicates an ID for identifying each item of measurement position information included in auscultation assisting information, and is assigned to each item of measurement position information.

For example, in auscultation assisting information, as shown in FIG. 12, items of measurement position information are represented by circles disposed on the basis of the contour 1060, the reference points 1061, and the reference lines 1062, and items of measurement position identification information may be indicated by alphabetical characters uniquely assigned to the individual circles.

The output control section 1022 may generate a projection image by superposing auscultation assisting information, such as that described above, on the patient image shown in FIG. 10.

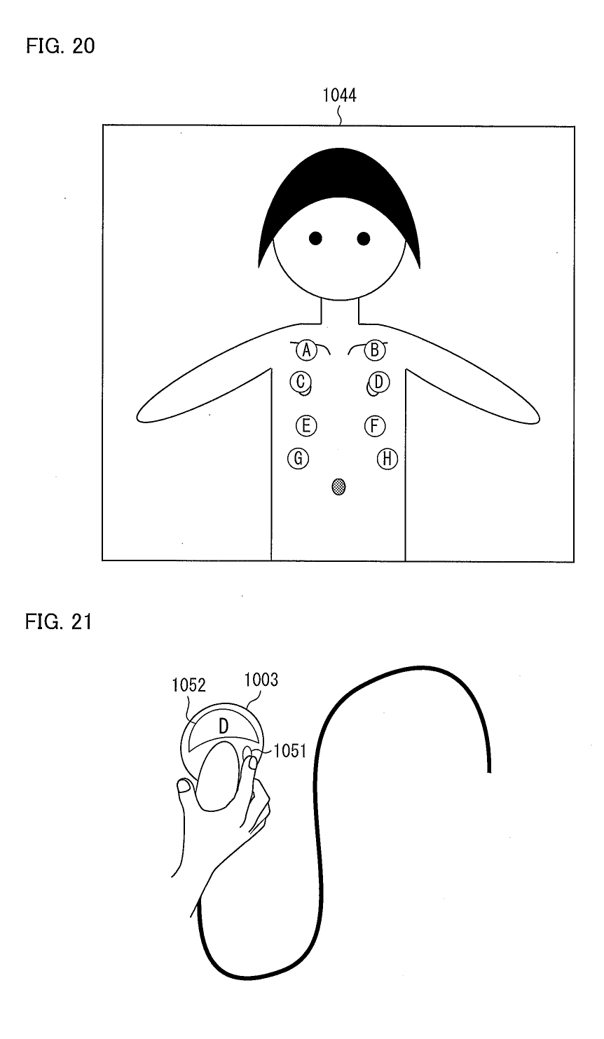

FIG. 20 illustrates another specific example of a projection image generated by the output control section 1022.

The auscultation assisting information includes, as shown in FIG. 20, measurement position information represented by circles and measurement position identification information (alphabetical character) assigned to each item of measurement position information. The output control section 1022 is able to generate a projection image 1044 shown in FIG. 20 by superposing the above-described auscultation assisting information selected by the auscultation-assisting-information selector 1021 on the patient image 1040 (FIG. 10) obtained from the imaging unit 1017.

Unlike the projection image 1043 shown in FIG. 13, in the projection image 1044, measurement order information is not provided, thereby allowing the operator U to start to conduct auscultation from a desired measurement position in a desired order. That is, the flexibility in the auscultation procedure is enhanced for the operator U.

In this modified example, however, since body sound information is not collected in a predetermined order, the information manager 1025 has to link measurement position information to body sound information in an approach different from the above-described approach.

FIG. 21 shows an outer appearance of the digital stethoscope 1003 used in this modified example. In this modified example, the digital stethoscope 1003 includes, as shown in FIG. 21, an operation button 1051 and a display unit 1052.

The operation button 1051 is a button for allowing an operator U to input and specify, among items of measurement position information included in auscultation assisting information output from the information management apparatus 1100, a measurement position at which auscultation will be conducted. The display unit 1052 displays an item of measurement position information corresponding to the measurement position selected by the operator U.

Auscultation assisting information output from the output control section 1022 of the information management apparatus 1100 is transferred from the information management apparatus 1100 to the digital stethoscope 1003 via the wireless communication unit 1016. The digital stethoscope 1003 outputs items of measurement position information included in the received auscultation assisting information for the operator U as options.

For example, when the auscultation assisting information shown in FIG. 20 is received, the digital stethoscope 1003 presents eight options A through H as items of measurement position information so that the operator U can select an option. For example, every time the operation button 1051 is pressed, measurement position identification information to be displayed in the display unit 1052 is changed, such as A, B, C, . . . H, A, and so on. While an item of measurement position identification information associated with a desired measurement position is being displayed in the display unit 1052, the operator U conducts auscultation so as to collect body sound information.

For example, while the digital stethoscope 1003 is in a state shown in FIG. 21, the operator U applies the digital stethoscope 1003 to the measurement position represented by measurement position identification information “D” shown in FIG. 20 and then collects body sound information. The digital stethoscope 1003 associates the measurement position identification information “D” to collected body sound information and sends the body sound information to the information management apparatus 1100.

The information management apparatus 1100 receives the measurement position identification information “D” and the body sound information via the wireless communication unit 1016. The event detector 1023 detects, as a specific event, that body sound information has been received. Upon detection of this event as a trigger, the information manager 1025 obtains auxiliary information necessary to specify a measurement position, that is, the measurement position identification information “D” received together with the body sound information.

On the basis of the obtained measurement position identification information “D” and auscultation assisting information which is currently output from the output control section 1022, the information manager 1025 is able to manage body sound information by linking measurement position information corresponding to the measurement position identification information “D” to the received body sound information.

As described above, in this embodiment, even if measurement order information is not included in auscultation assisting information, the information manager 1025 is able to manage body sound information by linking collected body sound information to a measurement position at which the sounds indicated by the body sound information have been collected. With this configuration, since a measurement order is not specified by measurement order information, the operator U is able to start to conduct auscultation from a desired measurement position in a desired order. That is, the flexibility in the auscultation operation is enhanced for the operator U.

### Second Mode of Second Embodiment

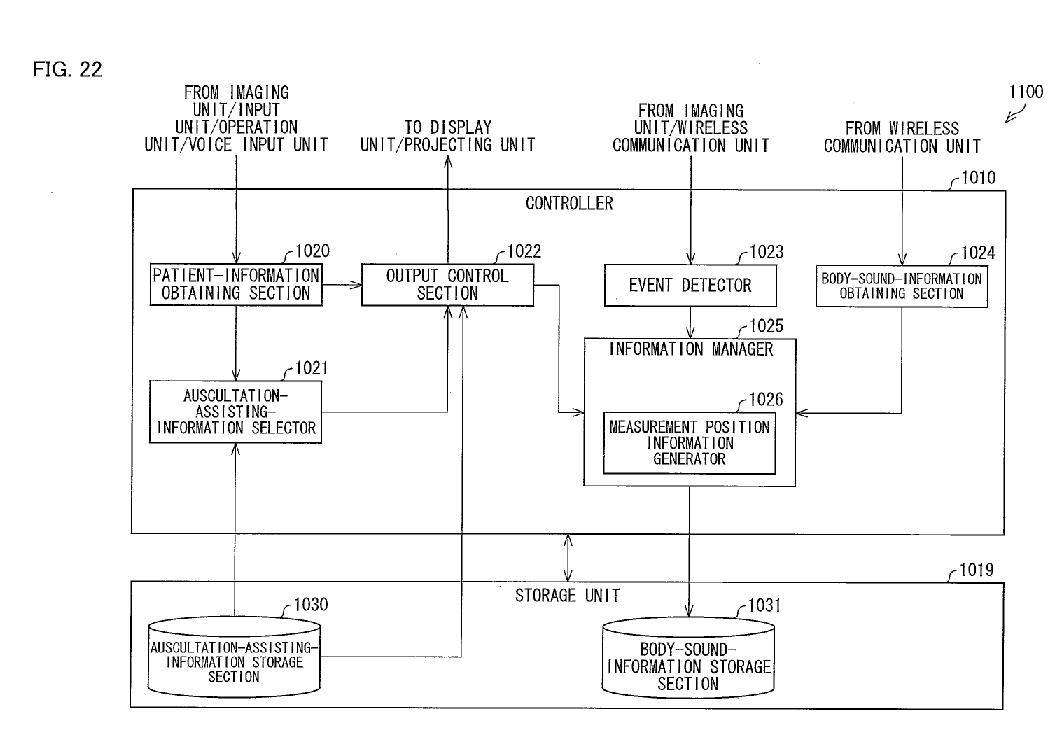

Another embodiment of the present invention of an information management apparatus of the present invention will be described below with reference to FIGS. 22 through 24. For the sake of convenience of description, elements having the same functions as those shown in the drawings discussed in the above-described first mode of the second embodiment are designated by like reference numerals, and an explanation thereof will thus be omitted.

In the above-described first mode of the second embodiment, for specifying a measured position of received body sound information, the information manager 1025 verifies a timing at which body sound information has been received against measurement order information included in auscultation assisting information or identification information related to the received body sound information.

However, the information manager 1025 is not restricted to the above-described configuration.

The information manager 1025 may specify a measurement position of received body sound information by analyzing a video image of the operator U conducting auscultation on a patient P.

### [Functional Configuration of Information Management Apparatus]

FIG. 22 is a functional block diagram illustrating the configuration of the major parts of the information management apparatus 1100 of this embodiment.

In the information management apparatus 1100 of the second mode of the second embodiment shown in FIG. 22 is different from that of the first mode of the second embodiment shown in FIG. 7 in that the information manager 1025 of the controller 1010 also includes a measurement position information generator 1026. As in the other blocks of the controller 1010, the measurement position information generator 1026 is a functional block which is also implemented as a result of, for example, a CPU, reading a program stored in a storage device (storage unit 1019) implemented by, for example, a ROM or an NVRAM, into, for example, a RAM, and executing the read program.

In this embodiment, the imaging unit 1017 captures an image of a patient P being subjected to auscultation by an operator U. Auscultation assisting information may be projected on the body surface of the patient P or on another light receiver, or may be displayed in another display unit. When the operator U starts to conduct auscultation by using the digital stethoscope 1003, the imaging unit 1017 captures an image of the patient P subjected to auscultation as a patient image.

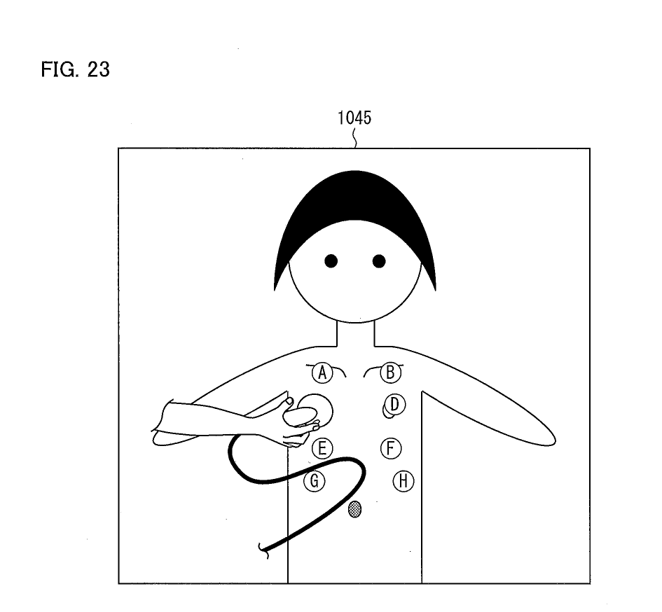

FIG. 23 illustrates a specific example of a patient image 1045 which indicates that a patient is being subjected to auscultation and which is obtained by the imaging unit 1017 in this mode of the embodiment. The patient image 1045 indicates that the operator U is applying the digital stethoscope 1003 to a predetermined measurement position. If auscultation assisting information can be projected on the body surface of the patient P, the projected auscultation assisting information is also contained in the patient image 1045. In order to take a clear image of the patient P and contact positions of the digital stethoscope 1003, it is preferable that the operator U conducts auscultation at a position at which the operator U is not in the way between these subjects and the imaging unit 1017 (for example, the position shown in FIG. 16).

In this mode of the embodiment, the event detector 1023 detects, as a specific event, that measurement for one measurement position is started or being performed. The event detector 1023 may detect such an event, on the basis of a signal indicating that auscultation is started or being conducted and transmitted from the digital stethoscope 1003 to the wireless communication unit 1016. Alternatively, the event detector 1023 may detect an event that auscultation is started or being conducted on the basis of the results of image recognition of a live view image (for example, the patient image 1045) obtained by the imaging unit 1017.

The measurement position information generator 1026 of the information manager 1025 obtains a frame of a live view image taken at a time at which a specific event was detected by the event detector 1023. This frame is the very image indicating that body sound information is being collected (a position of the digital stethoscope 1003) and this frame is auxiliary information necessary to specify a measurement position.

For example, it is now assumed that the event detector 1023 has detected that auscultation is started at a measurement position “C” in the auscultation assisting information shown in FIG. 20. Then, the measurement position information generator 1026 obtains, from a live view image obtained by the imaging unit 1017, a frame (for example, the patient image 1045 shown in FIG. 23) at a time at which the above-described event has been detected, as auxiliary information necessary to specify a measurement position.

The information manager 1025 utilizes the patient image 1045 obtained by the measurement position information generator 1026 as measurement position information, and links the patient image 1045 to body sound information which has been received since this event was detected.

With this configuration, body sound information is stored in the body-sound-information storage section 1031 by linking a patient image indicating a position of the digital stethoscope 1003 at which the sounds indicated by the body sound information have been collected to the body sound information. With this arrangement, even when reading the body sound information later, on the basis of the linked patient image, a physician D is able to recognize a measurement position at which these sounds have been collected.

Alternatively, when starting to conduct auscultation by using the digital stethoscope 1003, the operator U may operate the information management apparatus 1100 so that all measurement positions being subjected to auscultation will be recorded. For example, it may be possible that, when starting to conduct auscultation, the operator U touch a record start button displayed on a window of a digital stethoscope application. In response to this, the imaging unit 1017 starts recording video images. The measurement position information generator 1026 may extract, as measurement position information, a video frame taken at a time at which the event detector 1023 detected an auscultation start timing for each measurement position.

### Sixth Modified Example

It is not easy, however, to take a photo or a video image of a naked body of a patient and to link it to body sound information, that is, to store it in a non-volatile manner, in terms of the confidentiality of information and also by considering the patient's feelings.

Accordingly, in this mode of the embodiment, it is preferable that the measurement position information generator 1026 of the information manager 1025 specifies a measurement position from the obtained patient image 1045 by performing image recognition processing, and, instead of directly utilizing the patient image 1045, the measurement position information generator 1026 converts the patient image 1045 to measurement position information or measurement position identification information indicating the specified measurement position.

The measurement position information generator 1026 controls an image recognition processor (not shown) so as to recognize, in the patient image 1045, a measurement position of auscultation assisting information at which the digital stethoscope 1003 is being applied. For example, upon comparing the patient image 1045 with the auscultation assisting information shown in FIG. 20, the measurement position information generator 1026 is able to recognize that the digital stethoscope 1003 is being applied to a position of the circle to which the measurement position identification information “C” is appended. Accordingly, the measurement position information generator 1026 generates or obtains, not the patient image 1045 itself, but the measurement position identification information “C” or measurement position information (such as coordinate values) to which “C” is appended, as information for specifying a measurement position (auxiliary information).

Finally, the information manager 1025 is able to specify a measurement position on the basis of the auxiliary information. The information manager 1025 manages body sound information by linking measurement position information specified on the basis of auxiliary information generated or obtained by the measurement position information generator 1026 as described above (or auxiliary information itself) to body sound information received at a timing at which the patient image 1045 has been obtained.

With this arrangement, even when reading the body sound information later, on the basis of the linked measurement position information (or measurement position identification information), a physician D is able to recognize a measurement position at which the sounds indicated by the body sound information have been collected.

After the information manager 1025 has succeeded in linking a measurement position to body sound information, it is preferable that the measurement position information generator 1026 immediately deletes the patient image 1045 used for specifying the measurement position.

Alternatively, after performing image recognition processing on the patient image 1045 and specifying a measurement position, instead of directly utilizing the patient image 1045, the measurement position information generator 1026 may generate a model image distinctly indicating the specified measurement position.

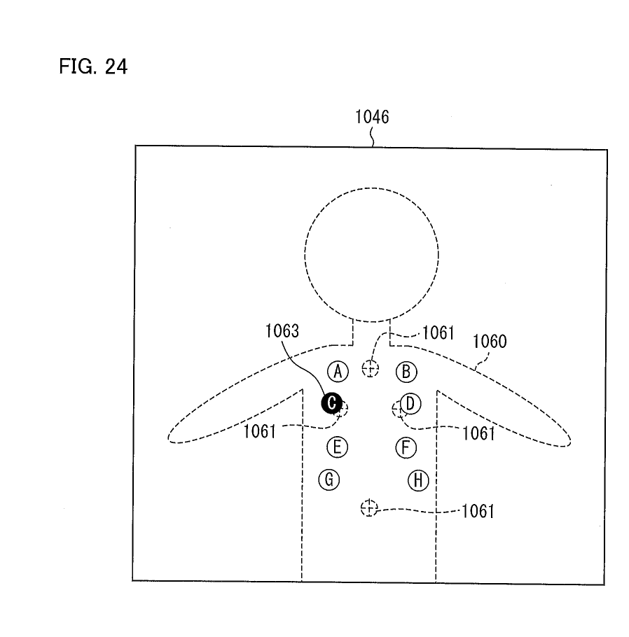

FIG. 24 illustrates a specific example of a model image indicating a measurement position generated by the measurement position information generator 1026.

A model image 1046 includes, as shown in FIG. 24, information indicating reference positions, such as a contour 1060 of a human, reference points 1061, and a reference line 1062. The model image 1046 also includes measurement position information 1063 distinctly indicating a measurement position. In this mode of the embodiment, the model image 1046 includes all items of measurement position information specified in the auscultation assisting information, and highlights the measurement position information 1063 distinctly indicating a measurement position. For example, the measurement position information 1063 is displayed in a color different from the color of the other items of measurement position information.

Alternatively, the measurement position information generator 1026 may generate the model image 1046 such that only measurement position information distinctly indicating a measurement position is displayed and the other items of measurement position information are not displayed.

The information manager 1025 links the model image 1046 generated by the measurement position information generator 1026 as described above to body sound information received at a timing at which the patient image 1045 has been obtained, and manages the body sound information.

With this arrangement, even when reading body sound information later, on the basis of a linked model image, a physician D is able to recognize a measurement position at which the sounds indicated by the body sound information have been collected.

### Third Mode of Second Embodiment

In the above-described embodiments, the information management apparatus 1100 includes the projecting unit 1014, and projects auscultation assisting information directly on a patient P. With this configuration, the operator U can intuitively understand measurement positions on the body surface of the patient P, and can be prevented from misrecognizing the measurement positions and the measurement order.

However, as shown in the example of the auscultation system 1200 in FIG. 8, if the operator U is a health care professional having medical expertise to some extent, it is not particularly necessary to present precise measurement positions to the operator U.

Accordingly, in this mode of the embodiment, a configuration in which auscultation assisting information is projected on a patient P by using the projecting unit 1014 is omitted, thereby further simplifying the configuration and the operation of the information management apparatus 1100.

The configuration of the information management apparatus 1100 of this mode of the embodiment is shown in FIG. 7. In this mode, however, the output control section 1022 outputs auscultation assisting information selected by the auscultation-assisting-information selector 1021, not to the projecting unit 1014, but to the display unit 1012 of the information management apparatus 1100. That is, in this mode of the embodiment, the output control section 1022 serves as a display controller that controls the content to be displayed in the display unit 1012.

This enables the operator U to check auscultation assisting information displayed in the display unit 1012 and to understand rough measurement positions and a measurement order before starting to use the digital stethoscope 1003, and then to conduct auscultation. Then, in a manner similar to that described above, the information management apparatus 1100 is able to manage body sound information by linking a measurement position to the body sound information.

In this mode of the embodiment, a configuration of “taking an image of a patient, estimating the body-build of the patient, and selecting precise auscultation assisting information which matches the body-build of the patient” can be omitted, thereby making it possible to further simplify the configuration and the operation of the information management apparatus 1100.

In this mode of the embodiment, instead of a patient image, the patient information obtaining section 1020 obtains, as patient information, information input by the operator U or information stored in the management server 1004.

In this mode of the embodiment, examples of the patient information obtained by the patient information obtaining section 1020 are information indicating whether the orientation of a patient to be subjected to auscultation is a front side or a back side, information concerning a disease of the patient to be diagnosed, and the medial history, age, gender, height, and weight of the patient.

In this mode of the embodiment, several patterns of auscultation assisting information according to the above-described patient information (or patient attributes specified on the basis of patient information) are stored in the auscultation-assisting-information storage section 1030.

The auscultation-assisting-information selector 1021 selects a pattern of auscultation assisting information that matches the patient information obtained by the patient information obtaining section 1020 or the patient attributes specified on the basis of the patient information.

The output control section 1022 outputs the pattern of auscultation assisting information selected by the auscultation-assisting-information selector 1021, not to the projecting unit 1014, but to the display unit 1012 of the information management apparatus 1100. For example, the output control section 1022 generates a video signal indicating the patterns of auscultation assisting information (Temp027 and Temp127) shown in FIG. 12 and the projection images 1043 and 1044 so that such items of information and the projection images 1043 and 1044 can be displayed in the display unit 1012, and outputs the generated video signal to the display unit 1012.

With this configuration, the configuration of the information management apparatus 1100 can be simplified by omitting high-load processing performed by, for example, an image recognition function and a projector function, and also, advantages substantially similar to those of the above-described modes of the embodiments can be obtained. That is, it is possible to allow an operator to perform measurement by using a digital stethoscope without using a special device involving a complicated operation and to easily link collected body sound information to measurement position information concerning the body sound information.

In the above-described embodiments, an example in which the information management apparatus 1100 of the present invention is applied to a smartphone has been discussed. However, the information management apparatus 1100 of the present invention may be implemented by various information processing apparatuses. For example, the information management apparatus 1100 of the present invention may be applicable to a personal computer (PC), an AV machine, such as a digital television, a notebook personal computer, a tablet PC, a cellular phone, a PDA (Personal Digital Assistant), a digital camera, and a digital video camera, though it is not restricted thereto.

### Modified Examples

In addition to linking of measurement information to body sound information collected by the digital stethoscope 1003, the information manager 1025 of the above-described first through third modes of the second embodiment may also link information which is input in relation to the body sound information to the body sound information by the operator U examining a patient on a face-to-face basis.

For example, it is now assumed that, in the auscultation system 1200 shown in FIG. 8, the operator U conducts auscultation by using the digital stethoscope 1003 in accordance with auscultation assisting information. Since the operator U is listening to collected body sound information while examining a patient on a face-to-face basis, the operator U may be able to diagnose the collected sounds to some extent.

For example, if some of the collected sounds are questionable (abnormal sounds or possible abnormal sounds), the operator U operates the digital stethoscope 1003 or the information management apparatus 1100 and inputs a check sign indicating that the collected body sound information includes questionable sounds.

Upon the event detector 1023 detecting that a check sign has been input into the obtained body sound information, the information manager 1025 links input information (such as a flag) indicating the collected body sound information includes questionable sounds to the obtained body sound information, and stores the obtained body sound information in the body-sound-information storage section 1031.

Then, the physician D being in a remote site accesses the management server 1004 and plays back the body sound information included in an electronic health record. In this case, on the basis of the input information linked to the body sound information, the management server 1004 is able to display a mark indicating that the sounds indicated by the body sound information are sounds questioned by the operator U, or to add a noticeable color to the measurement position of these sounds, and then presents this mark or color to the physician D. Alternatively, immediately before playing back the body sound information, the management server 1004 may issue warning sounds indicating that the sounds are sounds questioned by the operator U.

With this configuration, when the physician D listens to body sound information, attention, such as “these sounds are questionable sounds”, is given to the physician D. This makes the physician D consciously listen to the body sound information more carefully. As a result, even in a case in which the physician D being in a remote site has to make a final judgment of diagnosis due to a lack of medical experience of the operator U, it is less likely that the physician D will miss abnormality.

### Third Embodiment

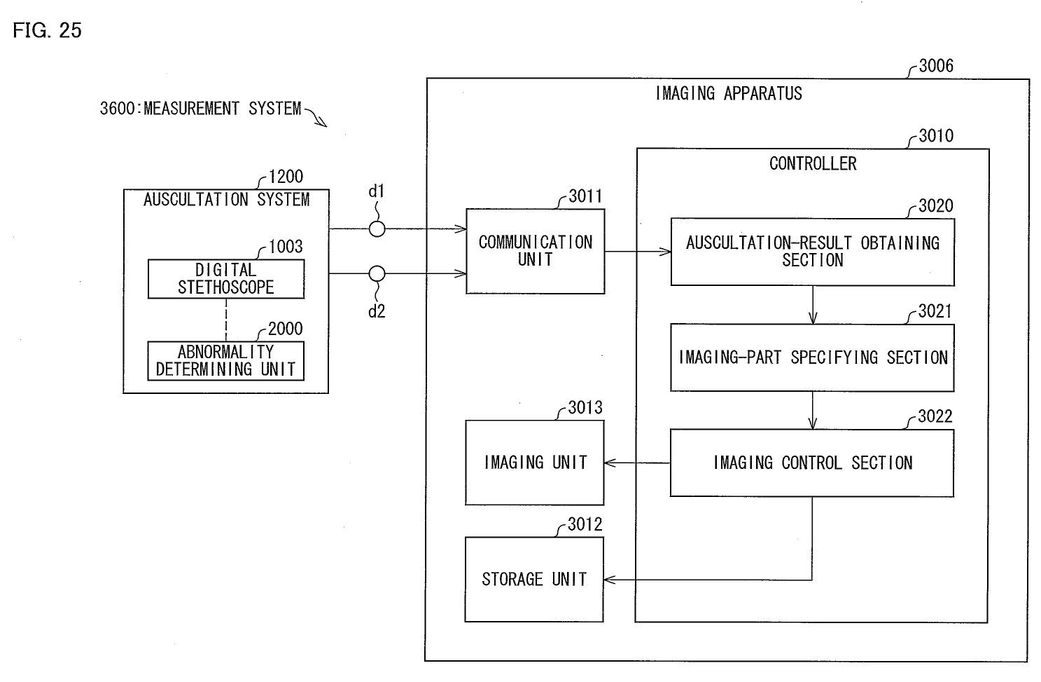

Another embodiment of the present invention will be described below with reference to FIG. 25. For the sake of convenience of description, elements having the same functions as those shown in the drawings discussed in the above-described embodiments are designated by like reference numerals, and an explanation thereof will thus be omitted.

### Background Art and Problems

PTL 5 discloses a medical image display system for creating and displaying a medical image in the following manner. A predetermined part of a body is imaged and image data indicating such an image part is obtained. Body sound measurement is then performed on the part of the body indicated in the image data. By associating measurement results of body sounds and the corresponding part of the body, a medical image is displayed.

In this configuration of the related art, however, imaging is performed without using measurement results of body sounds, and thus, it is not possible to perform imaging by focusing on a specific part in which an abnormality is occurring. Additionally, if there is no problem in the results of body sound measurement, the imaging operation performed on the body turns out to be a waste.

Accordingly, in this embodiment, a measurement system which performs medical imaging by considering measurement results of body sounds will be discussed.

### [Overview of Measurement System]

FIG. 25 is a block diagram illustrating an overview of a measurement system 3600 according to a third embodiment and the major parts of the configuration of an imaging apparatus 3006 forming the measurement system 3600.

The measurement system 3600 includes at least the digital stethoscope 1003 and the imaging apparatus 3006. The measurement system 3600 may also include the above-described auscultation system 1200 (FIG. 8) if necessary. That is, if necessary, the digital stethoscope 1003 and the imaging apparatus 3006 of the third embodiment are able to connect to various devices within the auscultation system 1200 in the above-described second embodiment so that they can communicate with such devices, and to operate in cooperation with the auscultation system 1200.

The digital stethoscope 1003 collects body sound information of a patient P. In this embodiment, the digital stethoscope 1003 serves as part of the auscultation system 1200 shown in FIG. 8.

The imaging apparatus 3006 images the patient P by using a suitable imaging unit so as to obtain image data. The image data obtained by the imaging apparatus 3006 is utilized by the operator U or the physician D as a medical image.

In this embodiment, the imaging apparatus 3006 is cooperated with the auscultation system 1200 shown in FIG. 8. The imaging apparatus 3006 is able to select optimal imaging processing for the patient P by considering auscultation results of the patient P obtained by the auscultation system 1200 and to perform the selected optimal imaging processing.

### [Configuration of Imaging Apparatus]

The imaging apparatus 3006 includes, as shown in FIG. 25, a communication unit 3011 which sends and receives information to and from the individual devices of the auscultation system 1200, a storage unit 3012 which stores therein various items of information processed by the imaging apparatus 3006, an imaging unit 3013 which images a patient, and a controller 3010 which centrally controls the individual elements of the imaging apparatus 3006.

The communication unit 3011 communicates with the individual devices of the auscultation system 1200 and receives auscultation results of the patient P obtained by the auscultation system 1200.

The storage unit 3012 stores therein, for example, image data obtained by the imaging unit 3013 and analysis result information d1 and body part information d2 obtained by the communication unit 3011.

The imaging unit 3013 images a body by using suitable means such as X rays, CT (Computed Tomography), MRI (Magnetic Resonance Imaging), magnetic measurement, bioelectric signals, ultrasound, or light, though the suitable means is not restricted thereto. In order to image a desired part of a patient P, the imaging unit 3013 may include a positioning mechanism for positioning an image sensor to an appropriate body part.

The controller 3010 includes, as functional blocks, an auscultation-result obtaining section 3020, an imaging-part specifying section 3021, and an imaging control section 3022.

The auscultation-result obtaining section 3020 controls the communication unit 3011 so that it can obtain auscultation results from the information management apparatus 1100. Auscultation results obtained by the auscultation-result obtaining section 3020 include at least two types of information. One type is analysis result information d1 indicating analysis results concerning body sound information collected by the digital stethoscope 1003. The other type is body part information d2 indicating a body part from which the body sound information is obtained. Specifically, the auscultation-result obtaining section 3020 obtains auscultation results at least indicating the presence or the absence of abnormality, which has been determined by the information measurement apparatus 1100 on the basis of the body sound information concerning the patient P, and a body part from which the body sound information has been collected.

The analysis result information d1 may include information determined by an abnormality determining unit (abnormality determining means) 2000, which is not shown. The abnormality determining unit 2000 is included in the information management apparatus 1100 and is connected to the digital stethoscope 1003 so that it can communicate with the digital stethoscope 1003. Upon receiving body sound information from the digital stethoscope 1003, the abnormality determining unit 2000 performs determination processing for determining whether or not this body sound information is an abnormality candidate (sounds which are highly likely to be abnormal sounds). That is, the information management apparatus 1100 may include the abnormality determining unit 2000 that analyzes body sound information collected by the digital stethoscope 1003. Alternatively, the abnormality determining unit 2000 may be included in another device (not shown) separately provided from the information management apparatus 1100 forming the above-described auscultation system 1200.

The abnormality determining unit 2000 determines by using a known technique whether or not body sound information is an abnormality candidate. As one example of a known technique, a determination may be made by comparing body sound information with sample data. More specifically, the following processing is an example of determination processing. The abnormality determining unit 2000 finds a similarity level between input body sound information with each item of sample data by using known waveform matching. If there is an item of sample data exhibiting a similarity level which is equal to or higher than a threshold, the abnormality determining unit 2000 determines that the body sound information is an abnormality candidate. If there is no such an item of sample data, the abnormality determining unit 2000 determines that the body sound information is normal (not an abnormality candidate).

In this embodiment, the imaging apparatus 3006 is connected to the information management apparatus 1100 of the second embodiment so that it can communicate with the information management apparatus 1100. The auscultation-result obtaining section 3020 obtains, via the communication unit 3011, determination results of the abnormality determining unit 2000 as analysis result information d1.

As discussed in the description of the second embodiment, body sound information is stored in and managed by the information management apparatus 1100 or the management server 1004 in the auscultation system 1200, and body part information indicating a body part from which body sound information has been collected is associated with the body sound information. The information management apparatus 1100 may receive input of body part information immediately before the operator U collects body sound information from the patient P by using the digital stethoscope 1003.

The body part information is measurement position information indicating a body part on the body surface of a patient P from which body sounds have been collected, and may be represented by a number appended to a circle shown in FIG. 13. The body part information is stored in the information management apparatus 1100 by associating it with body sound information, as described above.

The information management apparatus 1100 sends body part information associated with the body sound information, as body part information d2, together with analysis result information d1 concerning the body sound information, to the imaging apparatus 3006.

The auscultation-result obtaining section 3020 obtains auscultation results which have been sent as described above, that is, the analysis result information d1 and the body part information d2. The auscultation results obtained by the auscultation-result obtaining section 3020 are utilized for specifying a body part to be imaged by the imaging-part specifying section 3021.

The imaging-part specifying section 3021 specifies a body part to be imaged by the imaging unit 3013. The imaging-part specifying section 3021 specifies, as a part to be imaged, a position at which body sound information indicating the occurrence of abnormality or possible abnormality suggested by the analysis result information d1 has been collected. The imaging-part specifying section 3021 is able to specify a part to be imaged by using the body part information d2 obtained together with the analysis result information d1.

For example, it is now assumed that the analysis result information d1 obtained from the information management apparatus 1100 of the second embodiment includes determination results indicating “there is a possibility that body sounds are not normal (may be an abnormality candidate)” obtained by using the above-described known technique. In this case, the imaging-part specifying section 3021 refers to the body part information d2 obtained together with the analysis result information d1 so as to specify a part to be imaged. For example, if the body part information d2 indicates a number “3” shown in FIG. 13, the imaging-part specifying section 3021 specifies a part indicated by the circle to which the number “3” is appended (see FIG. 13) as a part to be imaged since there is a sign of abnormality in this part.

The imaging-part specifying section 3021 may be used, not only for selecting a part to be subjected to imaging, but also for refining a part to be subjected to precise imaging with higher resolution. For example, the imaging-part specifying section 3021 may determine that only the part “3” exhibiting a sign of abnormality will be imaged with a setting (for example, with higher resolution) different from a regular setting for the other parts.

The imaging control section 3022 sets various settings for the imaging unit 3013 on the basis of the body part specified by the imaging-part specifying section 3021, and then controls the imaging unit 3013 so that the body will be imaged. That is, the imaging control section 3022 performs imaging processing so that settings (imaging techniques) for the part specified by the imaging-part specifying section 3021 will be different from those for the other parts.

For example, if the part specified by the imaging-part specifying section 3021 is the part “3”, the imaging control section 3022 controls a positioning mechanism of the imaging unit 3013 so that the part “3” of the patient P will be precisely imaged. Alternatively, the imaging control section 3022 may set settings for the imaging unit 3013 so that imaging will be performed with higher precision only for the part “3”, and then perform imaging on the part “3” and the other parts.

Image data obtained by the imaging unit 3013 under the control of the imaging control section 3022 is stored in the storage unit 3012. In this case, when storing the image data, the imaging control section 3022 preferably associates the obtained image data with the corresponding analysis result information d1 and body part information d2. For example, the imaging control section 3022 associates the image data obtained by imaging the part “3” by the imaging unit 3013 with the analysis result information d1 indicating “there is a possibility that body sounds are not normal (may be an abnormality candidate)” and the body part information d2 indicating the part “3” and stores the image data in the storage unit 3012.

If a device including a function of analyzing body sound information and determining a disease is included in the auscultation system 1200, the analysis result information d1 may include information concerning the name of a disease if necessary. By informing the imaging apparatus 3006 of the name of a disease, the imaging control section 3022 is able to associate the name of a possible disease to obtained image data and store the image data in the storage unit 3012. If such image data is displayed in a display unit (not shown) together with the name of a disease and sound-type determination results, more detailed information can be provided to the physician D.

On the other hand, if the abnormality determining unit 2000 is able to determine, not only the presence or absence of abnormality, but also the degree (level) of abnormality, there may be some cases in which supplying of the degree (level) of abnormality to the imaging apparatus 3006 is more preferable than supplying of the name of a disease, as analysis result information d1. The reason for this is as follows. In the imaging apparatus 3006 of the present invention, it is possible to restrict parts of the patient P to be subjected to imaging processing to a minimal level. In this case, if the level of abnormality (the above-described determination results of the abnormality determining unit 2000) occurring in the patient P is supplied to the imaging apparatus 3006 as the analysis result information d1, the imaging-part specifying section 3021 is able to specify a part to be imaged in more details in accordance with the level of abnormality. More specifically, the imaging-part specifying section 3021 is able to specify the size of an area to be imaged in accordance with the level of abnormality. Although obtaining of a medical image with an unnecessarily large size is preferably avoided, an image size which is not sufficient to provide necessary information for a physician D to examine a patient P is pointless. Accordingly, it is desirable that, as auscultation results, in addition to body part information d2 indicating an abnormal part, analysis result information d1 indicating analysis results including the level of abnormality is supplied to the imaging apparatus 3006. Then, the imaging-part specifying section 3021 of the imaging apparatus 3006 preferably specifies the size of an area to be imaged in accordance with the level of abnormality.

The imaging control section 3022 controls the imaging unit 3013 in accordance with the size specified by the imaging-part specifying section 3021 so that it can obtain a medical image concerning a suitable part with a suitable size.

Hitherto, when obtaining a medical image, it is necessary that the operator U (or the physician D) of the imaging apparatus 3006 decide a part of a subject person (patient P) to be measured and operate the imaging apparatus 3006 so as to measure this part. In the measurement system 3600 of the present invention, on the basis of a part to be imaged specified by the imaging-part specifying section 3021 and the size of an area to be imaged determined by the imaging-part specifying section 3021, the imaging control section 3022 is able to position the imaging unit 3013 to a suitable location with respect to the subject person and to obtain a medical image. The obtained image data is then associated with body part information d2 and analysis result information d1 (the type and the level of abnormality) and is stored in the storage unit 3012. The stored image data is utilized as a medical image for conducting diagnosis by the physician D. Additionally, by managing the above-described attachment information associated with the image data, when reimaging of the same subject person becomes necessary after the first auscultation, the attachment information can be used as reference information. This also makes it possible to enhance the measurement precision in subsequent imaging processing. For example, the above-described attachment information can be utilized as follows. There may be a case in which a medical image obtained for the first time does not have information that the physician D has expected (the resolution is low, the imaging area is small, or an abnormal part has not been properly imaged). In this case, the imaging-part specifying section 3021 may make corrections by changing the part to be measured, the resolution, or the size of an area to be imaged from those specified in the previous processing so that image data having information desired by the physician D can be obtained.

As described above, in the measurement system 3600 of the present invention, the imaging apparatus 3006 is able to restrict parts of a patient P to be subjected to imaging processing to a minimal level by considering auscultation results output from the auscultation system 1200. That is, it is possible to implement the imaging apparatus 3006 and an imaging method that are capable of performing imaging processing which can provide sufficient information for a physician D to conduct diagnosis and which can also minimize the burden on a patient P. More specifically, on the basis of auscultation results, the imaging-part specifying section 3021 is able to decide to perform imaging only on a part in which the occurrence of abnormality (or possible abnormality) is recognized, or to perform imaging only on this part with higher resolution. For example, if the imaging unit 3013 is a mechanism which performs imaging with X rays, it is possible to reduce the radiation dose to which the patient P is exposed.

In this embodiment, the above-described auscultation system 1200 may be the body sound measurement system 100 of the first embodiment. In this case, the above-described digital stethoscope 1003 may be the digital stethoscope 1. The above-described information management apparatus 1100 may be the terminal device 30.

In this case, the terminal device 30 may include the above-described abnormality determining unit 2000, and the analysis result information d1 may include information determined by the abnormality determining unit 2000. The body part information d2 may be measurement position information as voice information input via the stethoscope 1. The terminal device 30 then supplies the analysis result information d1 and the body sound information d2 to the imaging apparatus 3006.

### [Examples of Implementations by Using Software]

The individual blocks of the terminal device 30, in particular, the main controller 37, may be implemented in the form of hardware logic, or may be implemented in the form of software by using a CPU in the following manner.

The individual blocks of the information management apparatus 1100, in particular, the patient information obtaining section 1020, the auscultation-assisting-information selector 1021, the output control section 1022, the event detector 1023, the body-sound-information obtaining section 1024, the information manager 1025, and the measurement position information generator 1026, may be implemented in the form of hardware logic, or may be implemented in the form of software by using a CPU in the following manner.

Additionally, the individual blocks of the imaging apparatus 3006, in particular, the auscultation-result obtaining section 3020, the imaging-part specifying section 3021, and the imaging control section 3022, may be implemented in the form of hardware logic, or may be implemented in the form of software by using a CPU in the following manner.

That is, the terminal device 30, the information management apparatus 1100, and the imaging apparatus 3006 each include a CPU (central processing unit) that executes commands of a control program which implements the individual functions, a ROM (read only memory) storing this program therein, a RAM (random access memory) loading this program, a storage device (recording medium), such as a memory, storing this program and various items of data therein, and so on. The object of the present invention may also be implemented by supplying a recording medium on which program code (an execution form program, an intermediate code program, and a source program) of the control program for each of the terminal device 30, the information management apparatus 1100, and the imaging apparatus 3006, which is software implementing the above-described functions, is recorded in a computer readable manner, to the terminal device 30, the information management apparatus 1100, and the imaging apparatus 3006, and by reading and executing the program code recorded on the recording medium by a computer (or a CPU or an MPU) of each of the terminal device 30, the information management apparatus 1100, and the imaging apparatus 3006.

As the above-described recording medium, for example, a tape type, such as magnetic tape or cassette tape, a disk type including a magnetic disk, such as a floppy (registered trademark) disk or a hard disk, and an optical disc, such as a CD-ROM, an MO, an MD, a DVD, or a CD-R, a card type, such as an IC card (including a memory card) or an optical card, or a semiconductor memory type, such as a mask ROM, an EPROM, an EEPROM (registered trademark), or a flash ROM may be used.

The terminal device 30, the information management apparatus 1100, and the imaging apparatus 3006 may be configured such that they are connectable to a communication network, and the above-described program code may be supplied to the terminal device 30, the information management apparatus 1100, and the imaging apparatus 3006 via the communication network. This communication network is not particularly restricted, and, for example, the Internet, an intranet, an extranet, a LAN, an ISDN, a VAN, a CATV communication network, a VPN (virtual private network), a public switched telephone network, a mobile communication network, a satellite communication work, etc. may be used. Additionally, a transmission medium forming this communication network is not restricted, and, for example, a wired transmission medium, such as IEEE1394, USB, power line communication, a cable TV line, a telephone line, or an ADSL circuit, or a wireless transmission medium, such as infrared, for example, IrDA or a remote controller, Bluetooth (registered trademark), 802. 11 radio, HDR (High Data Rate), a cellular phone network, a satellite circuit, or a terrestrial digital network, may be used. The present invention may also be realized in the form of a computer data signal embedded in a carrier wave in which the above-described program code is implemented through digital transmission.

### OTHER APPENDIXES

The present invention is not restricted to the above-described embodiments, and various modifications and changes may be made within the scope of the claims. An embodiment obtained by suitably combining technical means disclosed in the different embodiments is also encompassed in the technical scope of the present invention.

### Summary

The present invention may be described as follows.

An information management apparatus according to an embodiment of the present invention includes: obtaining means for obtaining body sound information obtained by a sound collector and position information indicating a position at which the body sound information has been obtained; and associating means for associating the body sound information and the position information obtained by the obtaining means with each other. The obtaining means obtains the position information as voice input into the sound collector.

With this configuration, in addition to body sound information, position information indicating a position at which the body sound information has been obtained is obtained as voice of a user by a sound collector. The obtaining means obtains such body sound information and position information. The associating means associates the body sound information and the position information obtained by the obtaining means with each other.

Accordingly, a user is able to input position information by a simple method, that is, voice input, thereby making it possible to reduce a burden imposed on the user caused by inputting position information. Additionally, a sound collector for obtaining body sound information can be utilized as an input device for inputting position information, thereby making it possible to eliminate the need to separately provide an input device for inputting position information.

The position information may preferably be obtained, together with the body sound information, as the same sound information, and the information management apparatus may preferably further include extracting means for extracting the position information and the body sound information from the sound information.

With this configuration, even if position information is obtained as voice superposed on body sound information, it is possible to separately extract the position information and the body sound information by using the extracting means.

The information management apparatus may preferably further include voice recognition means for performing voice recognition on the voice. The associating means may preferably associate the body sound information with position information obtained by performing voice recognition by the voice recognition means.

With this configuration, position information obtained as voice is subjected to voice recognition by the voice recognition means. Accordingly, position information can be obtained as characters or numbers or a combination thereof. Thus, it is possible to reduce the storage amount compared with a case in which position information is stored as voice. Additionally, position information can be displayed as characters or numbers, thereby enabling a user to easily check position information.

The information management apparatus may preferably further include: determining means for determining whether or not a position indicated by the position information obtained by performing voice recognition by the voice recognition means matches a predetermined position; and informing control means for causing, if the determining means has determined that the position indicated by the position information does not match the predetermined position, an informing unit to provide information that the position indicated by the position information does not match the predetermined position.

The information management apparatus may preferably further include an informing unit that provides, if a position indicated by the position information does not match the predetermined position, information indicating that the position indicated by the position information does not match the predetermined position.

With this configuration, if position information input by a user as voice does not match a predetermined position, it is possible to inform the user that position information input by the user as voice does not match the predetermined position, and to instruct the user to input correct position information.

The information management apparatus may preferably further include a display unit that displays a predetermined position of a body to be measured on which the sound collector is caused to abut.

With this configuration, it is possible to indicate a predetermined measurement position to a user.

An information management program for operating the information management apparatus and for causing a computer to function as each of the means, and a computer-readable recording medium on which the information management program is recorded are also encompassed within the technical scope of the present invention.

An information management method according to an embodiment of the present invention is an information management method for an information management apparatus. The information management method includes: a first obtaining step of obtaining body sound information obtained by a sound collector; a second obtaining step of obtaining position information indicating a position at which the body sound information has been obtained, as voice input into the sound collector; and an associating step of associating the body sound information obtained in the first obtaining step with the position information obtained in the second obtaining step.

With this configuration, in the first obtaining step, body sound information obtained by the sound collector is obtained, and in the second obtaining step, position information indicating a position at which the body sound information has been obtained is obtained. The order of the first obtaining step and the second obtaining step is not restricted, and the second obtaining step may be performed first, followed by the first obtaining step. Position information obtained in the second obtaining step is information input as voice via the sound collector.

In the associating step, the body sound information and the position information are associated with each other.

Accordingly, a user is able to input position information by a simple method, that is, voice input, thereby making it possible to reduce a burden imposed on the user caused by inputting position information. Additionally, a sound collector for obtaining body sound information can be utilized as an input device for inputting position information, thereby making it possible to eliminate the need to separately provide an input device for inputting position information.

A digital stethoscope according to an embodiment of the present invention includes: a first sound collecting unit that obtains body sounds; a second sound collecting unit that obtains, as voice information, position information indicating a position at which the body sounds have been obtained; and a transmitter that transmits body sound information indicating the body sounds and the position information to associating means for associating body sounds obtained by the first sound collecting unit with position information obtained by the second sound collecting unit.

With this configuration, the first sound collecting unit obtains body sounds, and the second sound collecting unit obtains, as voice information indicating user's voice, position information indicating a position at which the body sounds have been obtained. The first and second sound collecting units may be the same sound collecting unit or may be different sound collecting units.

The transmitter transmits body sound information indicating the body sounds and the position information to the associating means. The body sounds and the position information are associated with each other by this associating means.

Accordingly, a user is able to input position information by a simple method, that is, voice input, thereby making it possible to reduce a burden imposed on the user caused by inputting position information. Additionally, a sound collector for obtaining body sound information can be utilized as an input device for inputting position information, thereby making it possible to eliminate the need to separately provide an input device for inputting position information.

The second sound collecting unit may be a member different from a member of the first sound collecting unit, and may be suitable for obtaining voice.

With this configuration, the second sound collecting unit is configured such that it is suitable for obtaining voice, that is, position information, thereby making it possible to efficiently obtain position information.

An information management system including the above-described information management apparatus and the above-described digital stethoscope is also encompassed within the technical scope of the present invention.

The present invention may be described as follows.

In order to solve the above-described problems, the present invention provides an information management apparatus for managing body sound information collected by a stethoscope. The information management apparatus includes: subject information obtaining means for obtaining subject information concerning a subject from which body sound information is collected; an auscultation-assisting-information storage section that stores, according to the subject information, a plurality of patterns of auscultation assisting information each including at least one item of measurement position information indicating a measurement position to which a stethoscope will be applied; auscultation-assisting-information selecting means for selecting, from the plurality of patterns of auscultation assisting information stored in the auscultation-assisting-information storage section, one pattern of the auscultation assisting information corresponding to subject information obtained by the subject information obtaining means; output control means for generating an image signal from measurement position information included in the pattern of auscultation assisting information selected by the auscultation-assisting-information selecting means and for outputting the generated image signal; and information managing means for linking one item of the measurement position information output from the output control means to body sound information collected by the stethoscope. The information managing means specifies one item of the measurement position information on the basis of auxiliary information which has been obtained, as a trigger, upon the occurrence of a specific event while the image signal is being output.

With this configuration, on the basis of subject information obtained by the subject information obtaining means, the auscultation-assisting-information storage section is able to select auscultation assisting information suitable for a subject. The auscultation assisting information includes measurement position information indicating a measurement position to which a stethoscope will be applied.

The output control means outputs auscultation assisting information (in particular, measurement position information included in the auscultation assisting information) suitable for a subject as an image signal. With this arrangement, a measurement position to which a stethoscope will be applied for this subject is visualized. A user is then able to conduct auscultation suitable for the subject by referring to the visualized measurement position information.

As long as auscultation assisting information is being output by the output control means, the information managing means can assume that auscultation is being conducted in accordance with this auscultation assisting information. That is, since auscultation assisting information includes measurement position information, the information managing means can identify that the body sound information has been collected at a position indicated by one of the items of measurement position information included in the auscultation assisting information which is being output. Additionally, the information managing means is able to obtain auxiliary information necessary to specify measurement position information, as a trigger, upon the occurrence of a specific event while the auscultation assisting information (image signal) is being output.

Accordingly, concerning body sound information collected from a stethoscope, the information managing means is able to specify a position at which the body sound information has been collected, on the basis of the measurement position information included in the auscultation assisting information which is being output and the obtained auxiliary information.

Finally, the information managing means is able to link measurement position information indicating the specified measurement position to the body sound information.

The output control means outputs auscultation assisting information suitable for a subject as an image signal so that a user can visually check the image signal. When linking a measurement position to body sound information, the information managing means requires only the selected auscultation assisting information and auxiliary information which can be obtained by the information managing means, as a trigger, upon the occurrence of a specific event.

As a result, by using the information management apparatus of the present invention, it is possible to allow an operator to perform measurement by using a stethoscope without using a special device involving a complicated operation and to easily link collected body sound information to measurement position information concerning the body sound information.

The information management apparatus of the present invention may preferably include a projecting unit that projects, as an optical image, the image signal output from the output control means so that a user is able to visually check the optical image.

Additionally, in the information management apparatus, the auscultation-assisting-information storage section may preferably store the plurality of patterns of auscultation assisting information according to body-build of a subject. The subject information obtaining means may preferably obtain a subject image of the subject captured by an imaging unit. The auscultation-assisting-information selecting means may preferably estimate the body-build of the subject on the basis of the subject image and select a pattern of the auscultation assisting information corresponding to the estimated body-build of the subject.

With this configuration, a user does not have to perform a complicated input operation for selecting optimal auscultation assisting information, and instead, the user only has to make adjustment so that a subject will be contained in a capturing range of the imaging unit.

By visually checking a projected image, a user is able to correctly identify at which position a stethoscope should be applied in accordance with the body-build of the subject.

Additionally, in the information management apparatus, the optical image output from the projecting unit may preferably be projected on a body surface of the subject.

With this configuration, a user can intuitively understand measurement positions by the measurement position information projected on the body surface of the subject. By comparison with a mode in which measurement position information is displayed in a place other than the body of a subject, by using a mode in which measurement position information is displayed on the body surface of a subject, the user can be prevented from misrecognizing the measurement positions (or the measurement order). As a result, the frequency with which auscultation failures occur due to incorrect operations can be significantly reduced.

Additionally, projected auscultation assisting information is an item of information suitable for the body-build of a subject. Thus, projected measurement positions highly precisely reproduce portions of the patient to be subjected to auscultation.

Moreover, since the information management apparatus of the present invention displays measurement position information suitable for the body-build of a subject on the body surface of the subject, the user can intuitively understand measurement positions more suitable for the subject. Accordingly, even if the user does not have medical expertise, the user is able to perform correct auscultation.

In the information management apparatus of the present invention, in the auscultation assisting information, measurement order information indicating a measurement order may be appended to each item of the measurement position information. The output control means may generate an image signal from items of the measurement position information and associated items of the measurement order information. The information managing means may obtain, upon reception of the body sound information from the stethoscope as a trigger, the number of times the body sound information has been received since the image signal was output, as auxiliary information, and may specify one item of the measurement position information to be linked to the body sound information, on the basis of the number of times and the measurement order information.

With this configuration, as long as auscultation assisting information is being output by the output control means, the information managing means can assume that auscultation is being conducted in accordance with this auscultation assisting information. That is, since auscultation assisting information includes measurement position information and measurement order information, the information managing means can identify that the body sound information is sequentially collected at positions indicated by the individual items of measurement position information in the order indicated by the measurement order information included in the auscultation assisting information which is being output. Additionally, the information managing means is able to obtain auxiliary information necessary to specify measurement position information, that is, the number of times the body sound information has been received since the image signal was output, upon the occurrence of a specific event, as a trigger, indicating that the body sound information has been received while the auscultation assisting information (image signal) is being output.

The information managing means is able to specify measurement position information associated with the received body sound information, by referring to the number of times the body sound information has been received so far and the measurement order information included in the auscultation assisting information.

Alternatively, in the information management apparatus of the present invention, the information managing means may obtain, as the auxiliary information, a subject image, which is captured by an imaging unit, of the subject to which the stethoscope is being applied, upon reception of information, as a trigger, that collection of body sound information by the stethoscope is started or is being performed.

The subject image captured by the imaging unit is the very image indicating that body sound information is being collected (a position of the stethoscope). The subject image is auxiliary information necessary to specify a measurement position and is also measurement position information itself indicating a measurement position for the user.

The information managing means is able to utilize such a subject image for the management of body sound information, and to easily link a measurement position to body sound information.

In the information management apparatus, the information managing means may link the subject image to the body sound information.

With this configuration, the information managing means can manage body sound information by linking a subject image, which is measurement position information itself indicating a measurement position, to body sound information.

Alternatively, on the basis of a position of the stethoscope indicated in the subject image, the information managing means may specify, from auscultation assisting information which is being output as an image signal, one item of the measurement position information to be linked to the body sound information.

Alternatively, the information managing means may specify a measurement position, on the basis of a position of the stethoscope indicated in the subject image and auscultation assisting information which is being output as an image signal, generate a model image indicating the specified measurement position, and link the model image to the body sound information.

With this configuration, body sound information and a measurement position can be linked to each other and managed without the need to store a subject image together with body sound information.

It is not easy to take a photo or a video image of a naked body of a subject and to link it to body sound information, that is, to store it in a non-volatile manner, in terms of the confidentiality of information and also by considering the subject's feelings. With the above-described configuration, such a problem can be solved.

The information management apparatus of the present invention may further include a display unit that displays the image signal output from the output control means so that a user is able to visually check the image signal.

By checking an image displayed in the display unit, the user is able to understand at which position of a subject a stethoscope should be applied.

In order to solve the above-described problems, the present invention provides an information management method for managing body sound information collected by a stethoscope. The information management method includes: a subject information obtaining step of obtaining subject information concerning a subject from which body sound information is collected; an auscultation-assisting-information selecting step of selecting one pattern of auscultation assisting information corresponding to subject information obtained in the subject information obtaining step, from a plurality of patterns of auscultation assisting information stored in an auscultation-assisting-information storage section according to the subject information, each of the plurality of patterns of auscultation assisting information including at least one item of measurement position information indicating a measurement position to which a stethoscope will be applied; an output control step of generating an image signal from measurement position information included in the pattern of auscultation assisting information selected in the auscultation-assisting-information selecting step and outputting the generated image signal; and an information managing step of linking one item of the measurement position information output in the output control step to body sound information collected by the stethoscope. The information managing step specifies one item of the measurement position information on the basis of auxiliary information which has been obtained, as a trigger, upon the occurrence of a specific event while the image signal is being output.

In order to solve the above-described problems, the present invention provides a measurement system including: a digital stethoscope for conducting auscultation on a subject; one of the above-described information management apparatuses; and an imaging apparatus that performs imaging processing on the subject on the basis of auscultation results obtained by conducting auscultation by using the digital stethoscope and output from the information management apparatus. The information management apparatus further includes abnormality determining means for analyzing body sound information collected by the digital stethoscope. The imaging apparatus includes auscultation-result obtaining means for obtaining auscultation results which at least include information concerning the presence or the absence of abnormality which is determined by the abnormality determining means on the basis of the body sound information, and information concerning a part from which the body sound information has been collected, part specifying means for specifying a part for which the occurrence of abnormality has been determined, on the basis of the auscultation results obtained by the auscultation-result obtaining means, and imaging control means for performing imaging on a part specified by the part specifying means in a manner different from a manner for other parts so as to obtain image data concerning the subject.

With this configuration, the imaging apparatus is able to perform imaging processing by utilizing auscultation results output from the information management apparatus including the abnormality determining means. That is, the cooperation between measurements of auscultation sounds and imaging can be implemented. For example, imaging can be performed by focusing on a specific part in which an abnormality is observed in body sound information. Additionally, if there is no problem for a certain part in the results of auscultation sounds, a situation in which the imaging operation is uselessly performed for this part can be avoided.

The information management apparatus may be implemented in the form of a computer. In this case, a control program for implementing the information management apparatus in the form of a computer by causing the computer to function as each of the means of the information management apparatus and a computer-readable recording medium on which the control program is recorded are also encompassed within the scope of the present invention.

### INDUSTRIAL APPLICABILITY

In the present invention, it is possible to manage body sounds by associating body sounds obtained by a sound collector with a position at which the body sounds have been obtained. Accordingly, the present invention is applicable to a stethoscope and an information management apparatus that manages auscultation results obtained by using a stethoscope.

An information management apparatus of the present invention is capable of managing body sound information by linking body sound information measured and obtained by a digital stethoscope to a measurement position of a body at which measurement has been performed. Accordingly, the information management apparatus of the present invention can be widely used in a system in which body sound information is utilized by considering a measurement position. In particular, the information measurement apparatus of the present invention is suitably used in an auscultation system in which diagnosis and treatment is performed by conducting auscultation on collected body sound information by considering a measurement position.

### REFERENCE SIGNS LIST

1 stethoscope (sound collector, information management apparatus)
2 chestpiece (sound collector)
21 diaphragm face (first sound collecting unit)
22 vibration sensor (first sound collecting unit)
23 microphone (second sound collecting unit)
25 communication unit (transmitter)
30 terminal device (information management apparatus)
31 communication unit (receiver)
32 information separator (extracting means)
33 voice recognition section (voice recognition means)
34 position information determining section (determining means)
35 information manager (associating means)
36 output control section (informing control means)
37 main controller (obtaining means)
38 storage unit (memory unit)
39 display unit (informing unit)
40 speaker (informing unit)
50 subject (body)
100 body sound measurement system (information management system)
1001 clinic
1002 support center
1003 digital stethoscope (stethoscope)
1004 management server
1005 communication network
1010 controller
1011 input unit
1012 display unit
1013 operation unit
1014 projecting unit
1015 communication unit
1016 wireless communication unit
1017 imaging unit
1018 voice input unit
1019 storage unit
1020 patient information obtaining section (subject information obtaining means)
1021 auscultation-assisting-information selector (auscultation-assisting-information selecting means)
1022 output control section (output control means)
1023 event detector (event detecting means)
1024 body-sound-information obtaining section (body-sound-information obtaining means)
1025 information manager (information managing means/linking means)
1026 measurement position information generator (measurement-position-information generating means/auxiliary information obtaining means)
1030 auscultation-assisting-information storage section
1031 body-sound-information storage section
1040 patient image (subject image)
1041 patient image (subject image)
1043 projection image
1044 projection image
1045 patient image (subject image)
1046 model image
1050 installation table
1051 operation button
1052 display unit
1100 information management apparatus
1200 auscultation system
2000 abnormality determining unit (abnormality determining means)
3006 imaging apparatus
3010 controller
3011 communication unit
3012 storage unit
3013 imaging unit
3020 auscultation-result obtaining section (auscultation-result obtaining means)
3021 imaging-part specifying section (part specifying means)
3022 imaging control section (imaging control means)
3600 measurement system

1-26. (canceled)

27. An information management apparatus comprising:
obtaining means for obtaining body sound information obtained by a sound collector and position information indicating a position at which the body sound information has been obtained; and
associating means for associating the body sound information and the position information obtained by the obtaining means with each other,
wherein the obtaining means obtains the position information as voice input into the sound collector.

28. The information management apparatus according to claim 27, wherein:
the position information is obtained, together with the body sound information, as the same sound information; and
the information management apparatus further comprises extracting means for extracting the position information and the body sound information from the sound information.

29. The information management apparatus according to claim 27, further comprising:
voice recognition means for performing voice recognition on the voice,
wherein the associating means associates the body sound information with position information obtained by performing voice recognition by the voice recognition means.

30. The information management apparatus according to claim 29, further comprising:
determining means for determining whether or not a position indicated by the position information obtained by performing voice recognition by the voice recognition means matches a predetermined position; and
informing control means for causing, if the determining means has determined that the position indicated by the position information does not match the predetermined position, an informing unit to provide information that the position indicated by the position information does not match the predetermined position.

31. The information management apparatus according to claim 30, further comprising:
an informing unit that provides, if a position indicated by the position information does not match the predetermined position, information indicating that the position indicated by the position information does not match the predetermined position.

32. The information management apparatus according to claim 27, further comprising:
a display unit that displays a predetermined position of a body to be measured on which the sound collector is caused to abut.

33. A non-transitory computer-readable recording medium on which an information management program for operating the information management apparatus according to claim 27 and for causing a computer to function as each of the means is recorded.

34. An information management method for an information management apparatus, comprising:
a first obtaining step of obtaining body sound information obtained by a sound collector;
a second obtaining step of obtaining position information indicating a position at which the body sound information has been obtained, as voice input into the sound collector; and
an associating step of associating the body sound information obtained in the first obtaining step with the position information obtained in the second obtaining step.

35. A stethoscope comprising:
a first sound collecting unit that obtains body sounds;
a second sound collecting unit that obtains, as voice information, position information indicating a position at which the body sounds have been obtained; and
a transmitter that transmits body sound information indicating the body sounds and the position information to associating means for associating body sounds obtained by the first sound collecting unit with position information obtained by the second sound collecting unit.

36. The stethoscope according to claim 35, wherein the second sound collecting unit is a member different from a member of the first sound collecting unit, and is suitable for obtaining voice.

37. An information management system comprising:
an information management apparatus including
obtaining means for obtaining body sound information obtained by a sound collector and position information indicating a position at which the body sound information has been obtained, the position information being obtained as voice input into the sound collector and
associating means for associating the body sound information and the position information obtained by the obtaining means with each other; and
the stethoscope according to claim 35.
